(19) United States
(12) Patent Application Publication
(10) Pub. No.: US 2018/0153201 A1
(43) Pub. Date: Jun. 7, 2018

(54) AGROCHEMICAL-FREE SIRAITIA GROSVENORII EXTRACT, AND METHOD FOR PREPARING SAME

(71) Applicant: SARAYA CO., LTD., Osaka-shi, Osaka (JP)

(72) Inventors: Yuji MURATA, Osaka (JP); Takashi KUNISHIMA, Osaka (JP); Keika CHI, Osaka (JP); Yuta ITO, Osaka (JP)

(73) Assignee: SARAYA CO., LTD., Osaka-shi, Osaka (JP)

(21) Appl. No.: 15/577,721

(22) PCT Filed: May 27, 2016
(86) PCT No.: PCT/JP2016/065710

§ 371 Date: Nov. 28, 2017

(30) Foreign Application Priority Data
May 29, 2015 (JP) 2015-110825

(51) Int. Cl. A23L 27/10; A61K 47/46; A61K 8/9789; A61Q 19/00; C07J 17/00; A23L 33/105; A23L 2/60

(52) U.S. Cl. CPC A23L 27/11; A61K 47/46; A61K 8/9789; A23V 2002/00; C07J 17/005; A23L 33/105; A23L 2/60; A61Q 19/00

(57) ABSTRACT

A Siraitia grosvenorii extract preparation method is provided herein that removes an agrochemical selectively and efficiently from a Siraitia grosvenorii extract containing the agrochemical. The method further comprises collecting with high yield a Siraitia grosvenorii glycoside, a substance useful as a sweetener component.

### TECHNICAL FIELD

The present invention relates to a method for removing an agrochemical selectively and efficiently from an agrochemical-containing Siraitia Grosvenorii (Luo Han Guo) extract to prepare an agrochemical-free Siraitia Grosvenorii extract high in safety. More specifically, the invention relates to a method for preparing a Siraitia Grosvenorii extract, the method making it possible to recover, with a high yield, a Siraitia Grosvenorii glycoside useful as a sweetener constituent while an agrochemical is selectively and efficiently removed from an agrochemical-containing Siraitia Grosvenorii extract. The invention also relates to an agrochemical-free Siraitia Grosvenorii extract which has a sweet quality close to that of sugar and is high in quality and safety; and a sweetener composition including the Siraitia Grosvenorii extract.

### BACKGROUND ART

In recent years, as a sweetener alternative for sugar, many high sweetness sweeteners have been approved, and have widely been circulated in the market. The high sweetness sweeteners can be classified into artificial sweeteners (or synthetic sweeteners) and natural sweeteners.

Artificial high sweetness sweeteners include saccharin, aspartame, sucralose, acesulfame potassium, and advantame. Although the safety of all artificial sweeteners is guaranteed, the evaluation of the safety of the sweeteners are controversial.

Natural high sweetness sweeteners include sweeteners originating from plants, such as a licorice extract, a stevia extract, and a Siraitia Grosvenorii extract. It is stated that these natural sweeteners are historically old, and are high in safety for human bodies.

Out of these sweeteners, a Siraitia Grosvenorii extract is an extract of Siraitia Grosvenorii, which is a perennial plant of the family Cucurbitaceae for galenical preparations and is one special product around Guilin in Zhuang Autonomous Region, Guangxi, China. The extract has been widely used as a folk medicine in China from ancient times. Medical efficacies thereof extend against many cases, examples of the efficacies including the relief of sore throats and pain of throats, the curing of bronchitis and tonsillitis, cough suppression, expectoration, antipyresis, the promotion of gastrointestinal functions, stress reduction, diuresis, and constipation elimination. Moreover, in recent years, it is known that the Siraitia Grosvenorii extract has effects of the prevention of hypertension and diabetes mellitus, and anti-aging, and further has free-radical scavenging effect and anti-peroxide effect (Non-Patent Document 1).

Siraitia Grosvenorii extracts purified until the total amount of Siraitia Grosvenorii glycosides which are mogroside V, mogroside IV, 11-oxo-mogroside V, and siamenoside I turns to 33% or more by weight of the whole (the extracts are also called “high purity Siraitia Grosvenorii glycosides”) have sweet qualities very close to those of sugar in evaluations of basic performances of sweetness, which are “remaining flavor, over-rich flavor, peculiar flavor, astringency, stimulating flavor, refreshing flavor, mellowness, relish, and bitterness” (Non-Patent Document 2).

Artificial sweeteners are each produced by a chemical synthesis method. Thus, the sweeteners are generally high in quality stability, and further agrochemicals may hardly remain therein. However, the possibility is high that natural sweeteners originating from plants undergo agrochemical pollution in the step of breeding and cultivations of plants, the step of growth and development thereof, and the step of harvesting thereof. Thus, agrochemicals may not be sufficiently removed from the harvests.

Agrochemicals may also be used when Siraitia Grosvenorii are cultivated in agricultural farms. A problem of residual agrochemicals is not easily solved merely by countermeasures in the cultivation because even when agrochemical-management is made in the cultivation, Siraitia Grosvenorii may be polluted with agrochemicals used in neighboring farms when cultivated over a long term. When a Siraitia Grosvenorii extract is produced from harvested Siraitia Grosvenorii, in purifying and concentrating steps not only Siraitia Grosvenorii glycosides but also agrochemicals are concentrated, examples of the glycosides including mogroside V, mogroside IV, 11-oxo-mogroside V, and siamenoside I. Accordingly, agrochemicals undetectable at the harvesting time may be extracted from the purified Siraitia Grosvenorii extract. This Siraitia Grosvenorii extract may be used, as it is, as a sweetener. In many case, however, the extract is further purified and concentrated to be rendered “highly pure Siraitia Grosvenorii glycosides”, which include 33% or more of Siraitia Grosvenorii glycosides that are glycosides mogroside V, mogroside IV, 11-oxo-mogroside V and siamenoside I. However, also from commercially available “highly pure Siraitia Grosvenorii glycosides”, residual agrochemicals are frequently detected. According to conventional purifying methods, Siraitia Grosvenorii glycosides as described above are not easily separated completely from agrochemicals; thus, it is difficult to recover Siraitia Grosvenorii glycosides with a high yield while the agrochemicals are selectively and efficiently removed from the Siraitia Grosvenorii extract. The Siraitia Grosvenorii extract denotes any product extracted from fruits of Siraitia Grosvenorii by subjecting the fruits with water or any organic solvent, or a mixture of them.

As a conventional method for removing residual agrochemicals from a plant-extracted product, known is a method of bringing a plant-extracted product into contact with a porous adsorbing resin made of, for example, a three-dimensional copolymer made from styrene and divinylbenzene to adsorb agrochemicals remaining therein onto the adsorbing resin (Patent Document 1). However, this method has a problem that the species of removable agrochemicals are limited to organic chlorinated compound species.

As a method for removing residual agrochemicals and other pollutants included in an essential oil collected from a plant, known is also a method of using an activated carbon (Patent Document 2). As a method for removing residual agrochemicals effectively, suggested is also a method of bringing a plant-extracted product into contact with a carbon dioxide gas fluid in a supercritical or subcritical state (Patent Document 3). However, this method needs to be carried out, using a carbon dioxide gas supercritical processing apparatus; thus, this method cannot be easily carried out.

Patent Document 4 describes a method of bringing a Siraitia Grosvenorii extract into contact with an activated carbon, and at least one of a macro porous polymeric adsorbing resin and an ion exchange resin to purify the Siraitia Grosvenorii extract.

### PRIOR ART DOCUMENTS

### Non-Patent Documents

Non-Patent Document 1: “Foundations and Clinics”, 27 (8), 3159-3166 (1993)
No-Patent Document 2: “Sweet Properties of Siraitia Grosvenorii Glycosides, and Improvement thereof”, Journal of the Japanese Society for Food Science and Technology Nippon Shokuhin, vol. 53, No. 10, 2006, 10, 527-533

### Patent Documents

Patent Document 1: JP-A-2000-072790
Patent Document 2: JP-A-2010-116434
Patent Document 3: JP-A-2006-089414
Patent Document 4: JP-A-2014-506783

### SUMMARY OF THE INVENTION

### Problems to be Solved by the Invention

However, there remains a problem that as the removal ratio of pollutants becomes higher, a flavor component which is to be purified also becomes lower in yield.

An object of the present invention is to solve the problems in the prior art, specifically, provide a method for removing an agrochemical selectively and efficiently from a Siraitia Grosvenorii extract to prepare an agrochemical-free Siraitia Grosvenorii extract high in safety. More specifically, an object of the invention is to provide a method, for preparing a Siraitia Grosvenorii extract, making it possible to recover, with a high yield, Siraitia Grosvenorii glycosides useful as sweetener constituents while an agrochemical is selectively and efficiently removed from a Siraitia Grosvenorii extract. Another object of the invention is to provide an agrochemical-free Siraitia Grosvenorii extract which has a sweet quality close to that of sugar, and is high in safety; and a sweetener composition including the Siraitia Grosvenorii extract.

### Means for Solving the Problems

In order to solve the above-mentioned problems, the inventors have repeatedly made eager researches to find out that an activated carbon having specified physical properties are used to treat an agrochemical-containing Siraitia Grosvenorii extract, thereby making it possible to recover, with a high yield, Siraitia Grosvenorii glycosides (mogroside V, mogroside IV, 11-oxo-mogroside V, and siamenoside I) useful as sweetener constituents from the agrochemical-containing Siraitia Grosvenorii extract, and further remove, selectively and efficiently, agrochemicals remaining in the Siraitia Grosvenorii extract.

The present invention has been achieved on the basis of this finding, and includes the following embodiments:

### (I) Method for Preparing Agrochemical-Free Siraitia Grosvenorii Extract

(I-1) A method for preparing an agrochemical-free Siraitia Grosvenorii extract, comprising the step of treating an agrochemical-containing Siraitia Grosvenorii extract with an activated carbon to remove an agrochemical therein,

wherein the activated carbon used in the activated carbon treatment is at least one activated carbon selected from the group consisting of an activated carbon having an iodine adsorption amount of at least 1500 mg/g, and an activated carbon having a caramel decolorizing ability of at least 85%, and

the recovery ratio of Siraitia Grosvenorii glycosides according to the activated carbon treatment is at least 70%, the glycosides being mogroside V, mogroside IV, 11-oxo-mogroside V, and siamenoside I.

(I-2) The preparing method according to item (I-1), wherein the total amount of mogroside V, mogroside IV, 11-oxo-mogroside V, and siamenoside I comprised in the agrochemical-free Siraitia Grosvenorii extract is at least 33% by weight per 100% by weight of the agrochemical-free Siraitia Grosvenorii extract.

(I-3) The preparing method according to item (I-1) or (I-2), wherein the agrochemical is at least one, preferably two or more, more preferably six selected from the group consisting of dimethomorph, triadimenol, tebuconazole, difenoconazole, metalaxyl, and metalaxyl M.

The aspect of the invention can be rephrased in the same independent form as used for item (I-1) as follows:

“A method for preparing a Siraitia Grosvenorii extract containing no agrochemical (agrochemical-free Siraitia Grosvenorii extract), comprising the step of treating a Siraitia Grosvenorii extract containing an agrochemical (agrochemical-containing Siraitia Grosvenorii extract) with an activated carbon to remove an agrochemical therein,

wherein the activated carbon used in the activated carbon treatment is at least one activated carbon selected from the group consisting of an activated carbon having an iodine adsorption amount of at least 1500 mg/g, and an activated carbon having a caramel decolorizing ability of at least 85%, and

the agrochemical is at least one, preferably 2 or more, more preferably six selected from the group consisting of dimethomorph, triadimenol, tebuconazole, difenoconazole, metalaxyl, and metalaxyl M,

the recovery ratio of Siraitia Grosvenorii glycosides according to the activated carbon treatment is at least 70%, the glycosides being mogroside V, mogroside IV, 11-oxo-mogroside V, and siamenoside I.”

(I-4) The preparing method according to any one of items (I-1) to (I-3), wherein about the agrochemical-free Siraitia Grosvenorii extract, the area of a peak which is detected by subjecting the extract to an HPLC analysis under the following conditions and which is around a retention time of 16 minutes is 3% or less of the total area of all peaks detected thereby:

### [HPLC Conditions]

column: Kaseisorb LC ODS2000 (150 mm×4.6 mm I.D.) (manufactured by Tokyo Chemical Industry Co., Ltd.)

mobile phase: a mixed liquid of acetonitrile and water,
0 minutes→10 minutes (isocratic): 20% by volume of acetonitrile,
10 minutes→50 minutes (linear gradient): 20% by volume of acetonitrile→55% by volume thereof,

flow rate: 1 mL/minute,

column temperature: 40° C., and

detector: UV 203 nm.

### (II) Agrochemical-Free Siraitia Grosvenorii Extract

(II-1) An agrochemical-free Siraitia Grosvenorii extract,

the total amount of mogroside V, mogroside IV, 11-oxo-mogroside V, and siamenoside I comprised in the agrochemical-free Siraitia Grosvenorii extract being at least 33% by weight per 100% by weight of the agrochemical-free Siraitia Grosvenorii extract,

the area of a peak which is detected by an HPLC analysis of the extract under the following conditions and which is around a retention time of 16 minutes being 3% or less of the total area of all peaks detected thereby:

### [HPLC Conditions]

column: Kaseisorb LC ODS2000 (150 mm×4.6 mm I.D.) (manufactured by Tokyo Chemical Industry Co., Ltd.)

mobile phase: a mixed liquid of acetonitrile and water,
0 minutes→10 minutes (isocratic): 20% by volume of acetonitrile,
10 minutes→50 minutes (linear gradient): 20% by volume of acetonitrile→55% by volume thereof,

flow rate: 1 mL/minute,

column temperature: 40° C., and

detector: UV 203 nm.

(II-2) The agrochemical-free Siraitia Grosvenorii extract according to item (II-1), wherein the peak which is detected by the HPLC analysis and which is near the retention time of 16 minutes is a peak of a compound having a molecular weight of 358 daltons.

(II-3) The agrochemical-free Siraitia Grosvenorii extract according to item (II-1) or (II-2), wherein the agrochemical is dimethomorph, triadimenol, tebuconazole, difenoconazole, metalaxyl, and metalaxyl M.

The aspect of the invention can be rephrased in the same independent form as used for item (II-1) as follows:

“A Siraitia Grosvenorii extract containing no agrochemical (agrochemical-free Siraitia Grosvenorii extract),

the total amount of mogroside V, mogroside IV, 11-oxo-mogroside V, and siamenoside I comprised in the agrochemical-free Siraitia Grosvenorii extract being at least 33% by weight per 100% by weight of the agrochemical-free Siraitia Grosvenorii extract,

the area of a peak which is detected by an HPLC analysis of the extract under the following conditions and which is around a retention time of 16 minutes being 3% or less of the total area of all peaks detected thereby:

### [HPLC Conditions]

column: Kaseisorb LC ODS2000 (150 mm×4.6 mm I.D.) (manufacture by Tokyo Chemical Industry Co., Ltd.)

mobile phase: a mixed liquid of acetonitrile and water,
0 minutes→10 minutes (isocratic): 20% by volume of acetonitrile,
10 minutes→50 minutes (linear gradient): 20% by volume of acetonitrile→55% by volume thereof,

flow rate: mL/minute,

column temperature: 40° C., and

detector: UV 203 nm”.

(II-4) The agrochemical-free Siraitia Grosvenorii extract according to any one of items (II-1) to (II-3), which is prepared from an agrochemical-containing Siraitia Grosvenorii extract by the preparing method recited in any one of items (I-1) to (I-4)

### (III) Sweetener Composition, and Product Including the Same

(III-1) A sweetener composition, consisting of the agrochemical-free Siraitia Grosvenorii extract recited in any one of items (II-1) to (II-4), or comprising the agrochemical-free Siraitia Grosvenorii extract recited in any one of items (II-1) to (II-4) and an auxiliary sweetener constituent.

(III-2) The sweetener composition according to item (III-1), wherein the auxiliary sweetener constituent is at least one selected from the group consisting of monosaccharides disaccharides, oligosaccharides, sugar alcohols, non-saccharide sweeteners, and dietary fibers.

(III-3) A food or drink, quasi-drug, cosmetic product or medicine, comprising the sweetener composition recited in item (III-1) or (II-2)

### Effect of the Invention

The preparing method of the present invention makes it possible to recover, with a high yield, mogrosides (Siraitia Grosvenorii glycosides) that are mogroside V, mogroside IV, 11-oxo-mogroside V, and siamenoside I each useful as a sweetener constituent, and further remove one or more agrochemicals selectively and efficiently to give an agrochemical-free Siraitia Grosvenorii extract.

### BRIEF DESCRIPTION OF THE DRAWINGS

FIG. 1 shows HPLC chromatograms (in Experimental Example 4) of activated carbon untreated product; and a line represented by an arrow A is a chromatogram of a (post)-fraction of the activated carbon untreated product which is eluted out after a retention time of 27 minutes in a preparative HPLC of the product, and a line represented by an arrow B is a (pre)-fraction of the activated carbon untreated product which is eluted out before the retention time of 27 minutes in the preparative HPLC.

FIG. 2 shows HPLC chromatograms (in Experimental Example 4) of an activated carbon treated product (in Example 4); and a line represented by an arrow C is a chromatogram of a (post)-fraction of the activated carbon treated product which is eluted out after a retention time of 27 minutes in a preparative HPLC of the product, and a line represented by an arrow D is a (pre)-fraction of the activated carbon treated product which is eluted out before the retention time of 27 minutes in the preparative HPLC.

FIG. 3 shows a radar chart (in Experimental Example 4) obtained by evaluating sweet qualities of each of an activated carbon untreated product (raw material Siraitia Grosvenorii extract), a (post)-fraction of the activated carbon untreated product, and a (post)-fraction of an activated carbon treated product from the viewpoint of 8 items (bitterness, over-rich flavor, peculiar flavor, astringency, stimulating flavor, refreshing flavor, mellowness, and relish)

### MODE FOR CARRYING OUT THE INVENTION

### (1) Method for Preparing Agrochemical-Free Siraitia Grosvenorii Extract

The present invention is a method for preparing an agrochemical-free Siraitia Grosvenorii extract, and has the step of treating a Siraitia Grosvenorii extract which contains an agrochemical (agrochemical-containing Siraitia Grosvenorii extract) with an activated carbon to remove the agrochemical.

In this method, the agrochemical-containing Siraitia Grosvenorii extract used as a starting material is an extract from a Siraitia Grosvenorii fruit, and includes at least one Siraitia Grosvenorii glycoside selected from the group consisting of mogroside IV, mogroside V, siamenoside I and 11-oxo-mogroside V each represented by a chemical formula 1 illustrated below and shown in Table 1 described below. The agrochemicai-containing Siraitia Grosvenorii extract is preferably an extract including all of mogroside IV, mogroside V, siamenoside I and 11-oxo-mogroside V.

[Formula 1]

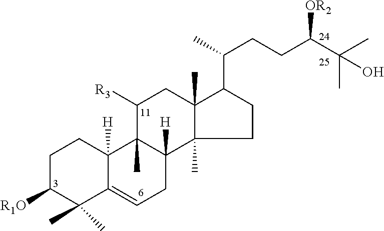

TABLE 1

| | R₁ | R₂ | R₃ |
|---|---|---|---|
| mogrosideIV | -Glc-Glc | -Glc-Glc | 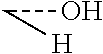 |
| mogroside V | -Glc-Glc | 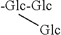 | 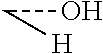 |
| siamenoside I | -Glc | 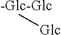 | 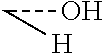 |
| 11-oxo-mogroside V | -Glc-Glc | 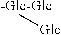 | ═O |

Glc; β-D-glucopyranosyl

The Siraitia Grosvenorii fruit used for the extraction may be a raw fruit or a dry-state fruit. The Siraitia Grosvenorii fruit is preferably a dry Siraitia Grosvenorii fruit from the viewpoint of extracting efficiency. The extraction may be made in the state that the shape of the (raw or dry) Siraitia Grosvenorii fruit is kept as it is. In the extraction, however, the fruit may be beforehand cut into slender pieces, crushed or pulverized.

A solvent used for the extraction may be any solvent capable of extracting the above-mentioned Siraitia Grosvenorii glycosides from a Siraitia Grosvenorii fruit. The solvent may be selected from the following to be usable: water; lower alcohols such as methanol, ethanol, butanol, and propanol; polyhydric alcohols such as glycerin, and propylene glycol; acetone, ethyl methyl ketone, ethyl acetate, methyl acetate, diethyl ether, cyclohexane, dichloromethane, 1,1,1,2-tetrafluoroethane, 1,1,2-trichloroethene, butane, propane, hexane, and other organic solvents; and any mixed liquid of water and one or more of these organic solvents. The organic solvents may be edible fats and oils. The extraction solvent is preferably water or a lower alcohol, in particular ethanol, or a mixed liquid of them, and more preferably water. The extraction may be performed in accordance with a usual method, and it does not matter whether the extraction is performed in the state of cold immersion or hot immersion, and performed in the state of still-standing or stirring.

In the case of using, as the extracting method, a supercritical extracting method or subcritical extracting method, examples of the extraction solvent include carbon dioxide and nitrous oxide.

When the agrochemical-containing Siraitia Grosvenorii extract used as the starting material is an extract containing an agrochemical and further containing the Siraitia Grosvenorii glycosides, this extract may be an extract subjected to the following besides the extracting treatment: solid-liquid separations such as filtration and centrifugation, adsorption, separation, concentration, drying, and other various treatments.

The agrochemical that is a target herein is at least one selected from the group consisting of dimethomorph (CAS No. 110488-70-5), triadimenol (CAS No. 55219-65-3), tebuconazole (CAS No. 107534-96-3), difenoconazole (CAS No. 1194146-68-3), metalaxyl (CAS No. 57837-19-1), and metalaxyl M (CAS No. 70630-17-0), which cause the problem of remaining in a Siraitia Grosvenorii extract. In other words, the agrochemical-containing Siraitia Grosvenorii extract which is a target of the present invention may be a Siraitia Grosvenorii extract containing at least one selected from the group consisting of the six agrochemicals. The agrochemical-containing Siraitia Grosvenorii extract may be an extract containing two or more of these six agrochemicals, plural ones thereof, or all the six agrochemicals.

These agrochemicals are each a compound having a distribution coefficient (LogPow) of 1.71 or more. Herein, the distribution coefficient of a target compound specifically means the 1-octanol/water distribution coefficient (LogPowₒₙ) thereof. When the target compound is added into two solvent phases (of 20 to 25° C.) of 1-octanol and water to make the two phases into an equilibrium state, the distribution coefficient more specifically means the concentration ratio of the compound between the two phases (1-octanol/water). The distribution coefficient may be gained by a flask method or an HPLC method. The distribution coefficient is a physicochemical index representing the hydrophobicity (solubility into lipid) of the compound. As the compound is higher in distribution coefficient, the compound is higher in hydrophobicity. It is known that as the compound is higher in hydrophobicity, the compound is higher in storability in a living body and is also stronger in toxicity. Thus, it can be stated that as the compound is higher in distribution coefficient, the compound is an agrochemical stronger in toxicity.

For reference, out of the six, about dimethomorph, tebuconazole, difenoconazole, metalaxyl, and metalaxyl N, the respective LogPow values are shown below.

TABLE 2

| | | Temperature | |
|---|---|---|---|
| | LogPow | (° C.) | Measuring method |
| Dimethomorph | 2.63 (E body) | 20 | Flask method |
| | 2.73 (Z body) | | |
| Triadimenol | 2.96 | — | Calculated value |
| Tebuconazole | 3.7 | 20 | Flask method |
| Difenoconazole | 4.4 | 25 | Flask method |
| Metalaxyl | 1.75 | 25 | Flask method |
| Metalaxyl M | 1.71 | 25 | HPLC METHOD |

See the homepage of Independent Administrative Institution Food and Agricultural Materials Inspection Center, “1. Agrochemical Abstract, Evaluation Document, and others” (https://www.acis/famic.go.jp/syouroku/index.htm).

In the case of gaining the LogPow value of triadimenol by making a calculation using the retention time of LC/MS out of the conditions described in paragraph [0064] (item (C) Agrochemical Analysis), and the respective LogPow values of tebuconazole and difenoconazole, which have a triazole structure as triadimenol, the value would be about 2.96. From these matters, the method of the present invention would make it possible to remove not only the above-mentioned specified agrochemicals but also compounds (including agrochemicals) each having a LogPow value (at 20 to 25° C.) of 1.71 or more.

Out of the six agrochemicals, three of triadimenol, tebuconazole and difenoconazole are each a compound having a triazole structure, more specifically a 1,2,4-triazole structure and having a distribution coefficient (LogPow) of 2.9 or more.

About the agrochemical content in the agrochemical-containing Siraitia Grosvenorii extract, the content of each of the above-mentioned agrochemicals is 0.01 ppm or more.

The activated carbon used in the activated carbon treatment in the present invention is at least one activated carbon selected from the group consisting of an activated carbon having an iodine adsorption amount of at least 1500 mg/g, and an activated carbon having a caramel decolorizing ability of at least 85%. The iodine adsorption amount is preferably 1.600 mg/g or more. The caramel decolorizing ability is preferably 90% or more, more preferably 95% or more. Activated carbons each having the iodine adsorption amount, and activated carbons each having the caramel decolorizing ability may each be used singly, or may be used in any combination of two or more thereof.

The iodine adsorption amount of an activated carbon is the amount (mg) of iodine (molecular diameter: about 5 Å, and radius: 2.29 Å) adsorbed onto the activated carbon per unit mass (1 g) thereof, and is a value corresponding to the specific surface area thereof.

A method for measuring the iodine adsorption amount of an activated carbon may be a method prescribed in “JIS K 1474: 2014, Activated Carbon Testing Method”, “7.1. Adsorbing Performances”, “7.1.2.2 Iodine Adsorbing Performance”,

### [Iodine Adsorption Amount Measurement]

Into 30 mL of distilled water is dissolved 25.0 g of potassium iodide, and thereto is added about 13 g of iodine to dissolve iodine. Thereto is added distilled water to adjust the volume of the whole into 1,000 mL to prepare an iodine solution having a concentration of 0.05 mol/L. Into a stopper-attached Erlenmeyer flask is put 1.0 g of a sample (activated carbon). Thereto is added 50 mL of the prepared iodine solution, and then the flask is wrapped with an aluminum foil piece. At a room temperature (of 20 to 3000), a shaker is used to shake the flask. A fraction of the content is then taken out at predetermined time-intervals, and shifted into a 50 mL centrifuging sedimentation tube. A centrifugal separator is used to produce a precipitation at 3,000 rpm for 15 minutes. From the inside of the tube, 10 mL of the resultant supernatant is precisely collected, and titrated with a 0.1 mol/L sodium thiosulfate solution. When the yellowish color of iodine becomes thin, 1 ml of a starch solution (10 g/L) as an indicator is added thereto. Furthermore, the indicator-added solution is continuously titrated. A time when the bluish color of the indicator vanishes is determined to be the end point of the titration. From the concentration of remaining iodine (remaining iodine concentration), the Iodine adsorption ability of the sample carbon is gained.

The remaining iodine concentration (g/L) is calculated out in accordance with the following expression:

I_{N}=K×f×12.69×1/10 [Math. 1]

I_{N}: Remaining iodine concentration (g/L)

K: Volume (mL) of 0.1 mol/L sodium thiosulfate solution used for titration (mL)

f: Factor of 0.1 mol/L sodium thiosulfate solution

12.69: Iodine equivalent quantity (mg/mL) of 1 mL of 0.1 mol/L sodium thiosulfate solution (mg/mL)

10: Collected supernatant volume (mL)

The iodine adsorption amount (mg/g) of the sample per unit mass thereof is calculated out in accordance with the following expression:

A₁=[(10×f′−K×f)×12.69×5]/S [Math. 2]

A₁: Iodine adsorption amount (mg/g) of sample per unit mass thereof (mg/g)

S: Sample mass (g)

f′: Factor of 0.05 mol/L iodine solution

K: Volume (mL) of 0.1 mol/L sodium thiosulfate solution used for titration (mL)

f: Factor of 0.1 mol/L sodium thiosulfate solution

12.69: iodine equivalent quantity (mg/mL) of 1 mL of 0.1 mol/L sodium thiosulfate solution (mg/mL)

A method for measuring the caramel decolorizing performance of an activated carbon may be a method prescribed in “JIS K 1474: 2014, Activated Carbon Testing Method”, “7.2 Caramel Decolorizing Performance”.

### [Caramel Decolorizing Performance Measurement]

A sample (activated carbon) is beforehand dried in a temperature-constant drying machine of 115±5° C. temperature, and then naturally cooled to room temperature in a desiccator. To 150 mg of the resultant sample is added 50 mL of a caramel testing liquid, and a shaker is used to shake the sample at a room temperature (of 20 to 30° C.) for 30 minutes. Thereafter, the resultant is filtrated through a filtrating paper piece. A 20 mL initial fraction of the resultant filtrate is thrown away, and the absorbance of a subsequent fraction thereof is measured at 430 nm. About 50 mL of a caramel testing liquid, a blank test is separately made by the same operations as when and after the shaking operation is made. From the absorbance of the filtration, and that of the caramel testing liquid (blank test), the caramel decolorizing performance is gained.

The used caramel testing liquid is a liquid prepared by the following method:

### (Caramel Testing Liquid Preparation)

Into an Erlenmeyer flask of 500 ml volume is put 60 g of sucrose dried in a desiccator for 24 hours or longer. Thereto is added 240 mL of water to dissolve sucrose at a room temperature (of 20 to 30° C.). Thereto is added 25 mL of a sulfuric acid liquid (sulfuric acid 1: water 4), and the resultant is immersed in a water bath adjusted to a temperature of 80±1° C. While the solution in the Erlenmeyer flask is stirred, the temperature thereof is raised to a temperature close to 800° C. Furthermore, the solution is kept in a state of 80±1° C. temperature for 30 minutes. From the water bath, the Erlenmeyer flask is taken out, and immediately 50 mL of a sodium hydroxide solution (200 g/L) is added thereto. The Erlenmeyer flask is immediately put into a boiled water bath to be heated for 15 minutes. The Erlenmeyer flask is taken out from the boiled water bath, and then allowed to stand still at room temperature all night. Thereto is then added a sulfuric acid liquid (sulfuric acid 1:water 10), or a sodium hydroxide solution (100 g/L) to adjust the pH of the resultant to 7.0±1.0. The entire volume of the solution in the Erlenmeyer flask is sifted into a 500 mL flask. Water is then added to the flask till its marked line for 500 mL.

After a while, the prepared raw caramel liquid is diluted with water into a ratio by volume of liquid/water that is 1/20, and then allowed to stand still for 30 minutes. A photometer is then used to measure the absorbance thereof at a wavelength of 430 nm. The dilution ratio of the resultant liquid is adjusted to make the absorbance thereof consistent with that of a chromaticity standard solution described below. The prepared solution is used as a caramel testing liquid.

### [Chromaticity Standard Solution]

Potassium dichromate is pulverized in an agate mortar, and dried in a constant-temperature drying machine adjusted to a temperature of 105±+5° C. for 3 to 4 hours. This salt is then naturally cooled in a desiccator. Therefrom, a fraction of 0.310 g weight is weighed out. Water is added thereto to dissolve the salt. The whole volume of this solution is shifted into a flask, and water is then put into the flask till its marked line to prepare a chromaticity standard solution. A photometer is used to measure the absorbance of this solution at a wavelength of 430 nm.

The caramel decolorizing performance (%) of the sample (activated carbon) is calculated out in accordance with an expression described below. The resultant value is rounded off to the first digit after the decimal point thereof. Thus, the value is represented as a value having a number of the first digit after the decimal point.

D=1−(E′/E)×100(%) [Math. 3]

D: Caramel decolorizing performance (%)

E′: Absorbance of liquid (filtrate) decolorized by sample

E: Absorbance (of caramel testing liquid) in blank test

About the activated carbon used in the present invention, the shape and the size thereof are not particularly limited. Any one of a powdery activated carbon, and granular activated carbons (pulverized, finely granular and granulated activated carbons), and spherical and fibrous activated carbons can be used as activated carbon. Out of these activated carbons, powdery and granular activated carbons are preferably usable since the activated carbons produce a great advantage effect of removing residual agrochemicals from a Siraitia Grosvenorii extract, and are further excellent in handleability, adsorbing efficiency, economy and others. The activated carbon is preferably an activated carbon selected from the group consisting of a granular activated carbon having an iodine adsorption amount of at least 1500 mg/g, and a powdery activated carbon having a caramel decolorizing ability of at least 85%.

The granular activated carbon denotes an activated carbon in the form of granules which usually have a size that is a diameter or major diameter of 0.15 mm or more. Examples thereof include crushed carbon, finely granular carbon, and granulated carbon. The activated carbon is preferably crushed carbon prepared by crushing coconut shells or a woody material (such as sawdust or woody chips). Although the crushed carbon is in a granular form, the shape and the size are not constant. The powdery activated carbon denotes an activated carbon in the form of powders which usually have a size that is a diameter or major diameter smaller than 0.15 mm, and is preferably a powdery activated carbon having a diameter of 0.10 mm or less.

Corresponding to the species of the activated carbon, the particle size distribution thereof may widen below and above a diameter of 0.15 mm (for example, a range from 0.1.3 to 0.16 mm). In this case, the form of the activated carbon can be judged by analyzing whether or not the particle size distribution by weight is 50% or more. When the particle size distribution by weight of a fraction of an activated carbon which has a size of 0.15 mm or more is 50%; or more, the activated carbon can be judged to be a “granular activated carbon”. Conversely, when the particle size distribution by weight of a fraction of an activated carbon which has a size less than 0.15 mm is 50% or more, the activated carbon can be judged to be a “powdery activated carbon”.

The origin of the activated carbon used in the present invention is not particularly limited. Thus, the activated carbon may be an activated carbon produced using, as a raw material, any one of coconut shells, woody materials (such as sawdust and woody chips), charcoal, and petroleum. In particular, an activated carbon obtained using, as a raw material, coconut shells or a woody material (such as sawdust or woody chips) is favorably used to prepare a Siraitia Grosvenorii extract used in a sweetener composition for food from the viewpoint of safety and the prevention of environmental pollution.

The activated carbon used in the present invention is not limited. Examples thereof include a product FG-3 manufactured by Japan Enviro Chemicals, Ltd. as a granular activated carbon having an iodine adsorption amount of at least 1500 mg/g; and products FP-1, FP-3 and FP-6 each manufactured by Japan Enviro Chemicals, Ltd., and a product KURARAY COAL PK-D manufactured by Kuraray Chemical Co., Ltd. as powdery activated carbons having a caramel decolorizing ability of at least 85%.

For the activated carbon treatment applied to an agrochemical-containing Siraitia Grosvenorii extract, for example, the following are adoptable: a method of adding the activated carbon to the agrochemical-containing Siraitia Grosvenorii extract, stirring the mixture to cause the agrochemicals in the extract to be adsorbed on the activated carbon, and then separating the activated carbon from the agrochemicals (batch method); and a method of passing the agrochemical-containing Siraitia Grosvenorii extract through a column filled with the activated carbon to cause the agrochemicals in the extract to be adsorbed on the activated carbon in the column, and then collecting the filtrate (column method). The agrochemical-containing Siraitia Grosvenorii extract supplied for the activated carbon treatment is preferably a liquid in order for the method to produce the efficiency of the working, and a greater advantageous effect. The agrochemical-containing Siraitia Grosvenorii extract may be optionally diluted with water to be used.

When from an agrochemical-containing Siraitia Grosvenorii extract the agrochemicals are removed by the column method, appropriate selection and adjustments may be made about the species of the used activated carbon, the amount of the activated carbon filled into the column, the flow rate of the agrochemical-containing Siraitia Grosvenorii extract into the activated-carbon-filled column, and others in accordance with the species and the amount of Siraitia Grosvenorii glycosides included in the agrochemical-containing Siraitia Grosvenorii extract, the species and the amount of the agrochemicals contained in the agrochemical-containing Siraitia Grosvenorii extract, and other factors. In the case of using, for example, a granular activated carbon as the activated carbon, the granular activated carbon is used in an amount desirably from 100 to 5000 parts by weight, more preferably from 500 to 2000 parts by weight for 100 parts by dry weight of the agrochemicai-containing Siraitia Grosvenorii extract. In the case of using, for example, a powdery activated carbon as the activated carbon, the powdery activated carbon is used in an amount desirably from 0.1 to 150 parts by weight, more preferably from 5 to 50 parts by weight for 100 parts by dry weight of the agrochemical-containing Siraitia Grosvenorii extract.

When a granular activated carbon is used as the activated carbon, for example, the following method makes it possible to remove agrochemicals from the agrochemical-containing Siraitia Grosvenorii extract to prepare a Siraitia Grosvenorii extract which does not contain the agrochemicals:

(1) A necessary amount of the granular activated carbon is filled into a column, and deionized water is caused to flow into the column from the top to the bottom thereof to wash away fine powder of the activated carbon, and others.

(2) Water is beforehand added into the granular-activated-carbon-filled column prepared as described above, and then an aqueous solution of the Siraitia Grosvenorii extract, the concentration of which is adjusted into a range from 5 to 20% by weight/volume, more preferably from 5 to 10% by weight/volume, is poured into the column.

(3) Deionized water is poured into the column. The resultant flowing Siraitia-Grosvenorii-extract-containing solution is fractionized.

(4) About each of fractional solutions obtained by the fractionization, for example, a thin layer chromatography or HPLC is used to check whether or not each of mogroside V, mogroside IV, 11-oxo-mogroside V, and siamenoside I is present. Out of the entire fractions, respective fractions containing these Siraitia Grosvenorii glycosides are each collected.

(5) The collected fractions, which contain the respective Siraitia Grosvenorii glycosides, are combined with each other to prepare an agrochemical-free Siraitia Grosvenorii extract.

Mogroside V, out of the Siraitia Grosvenorii glycosides, can be analyzed (qualitatively and quantitatively) according to an HPLC apparatus and conditions described in item (A) given below. Each of the Siraitia Grosvenorii glycosides other than mogroside V (Mogroside IV, 11-oxo-mogroside V, or siamenoside I) can be analyzed (qualitatively and quantitatively) according to an HPLC apparatus and conditions described in item (B) given below.

### (A) Mogroside V Analysis

HPLC apparatus: Prominence LC-20AD (manufactured by Shimadzu Corp.),

LC Column: Shodex Asahipak NH2-50 4E (manufactured by Show Denko K.K.; 250 mm×4.6 mm I.D.), and

LC conditions:
injected amount: 20 μL,
column temperature: 40° C.,
mobile phase: 74% by volume of acetonitrile and 26% by volume of water,
flow rate: 1.0 mL/minute, and
detecting wavelength: UV 203 nm.

### (B) Mogroside IV, 11-Oxo-mogroside V, or Siamenoside I Analysis

HPLC apparatus: Prominence IC-20AD (manufactured by Shimadzu Corp.),

LC column: Kaseisorb LC ODS 2000 (150 mm×4.6 mm I.D.) (manufactured by Tokyo Chemical Industry Co., Ltd.), and

LC conditions:
injected amount: 20 μL,
column temperature: 40° C.,
mobile phase: phase A: water, and phase B: acetonitrile,
0 minutes→10 minutes (isocratic): 80% by volume of phase A, and 20% by volume of phase B,
10 minutes→50 minutes (linear gradient): “80% by volume of phase A, and 20% by volume of phase B”→“45% by volume of phase A, and 55% by volume of phase B”,
flow rate: 1.0 mL/minute, and
detecting wavelength: UV 203 nm.

In the above-mentioned process, one or more Siraitia Grosvenorii glycosides (mogroside V, mogroside IV, 11-oxo-mogroside V, and/or siamenoside I) contained in the flowing-out fractions from the column may be checked by detecting a light refractivity change based on a change in component-composition of the column flowing-liquid, using a differential refractive index detector.

When from an agrochemical-containing Siraitia Grosvenorii extract the agrochemicals are removed by the batch method, appropriate selection and adjustments may be made about the species and the amount of the activated carbon to be used, the stirring period, and others in accordance with the species and the amount of the Siraitia Grosvenorii glycosides included in the agrochemical-containing Siraitia Grosvenorii extract, the species and the amount of the agrochemicals contained in the agrochemical-containing Siraitia Grosvenorii extract, and other factors. In the case of using, for example, a granular activated carbon as the activated carbon, the granular activated carbon is used in an amount desirably from 100 to 5000 parts, more preferably from 500 to 2000 parts by weight for 100 parts by dry weight of the agrochemical-containing Siraitia Grosvenorii extract. In the case of using, for example, a powdery activated carbon as the activated carbon, the powdery activated carbon is used in an amount desirably from 0.1 to 150 parts, more preferably from 5 to 50 parts by weight for 100 parts by dry weight of the agrochemical-containing Siraitia Grosvenorii extract. In the case of using, for example, a powdery activated carbon as the activated carbon, it is desired to add, into any container, the agrochemical-containing Siraitia Grosvenorii extract, and a necessary amount of the powdery activated carbon, and then stir the resultant for about 5 minutes to 2 hours, more preferably 30 to 60 minutes. Conditions for the temperature are not particularly limited. The temperature may be from 25 to 6000. It is preferred that the water content in the agrochemical-containing Siraitia Grosvenorii extract is from 1 to 50% by weight/volume, preferably from 5 to 10% by weight/volume. It is desired to add water to the agrochemical-containing Siraitia Grosvenorii extract to adjust the water content into the above-mentioned water content value as the need arises. It is further desired to stir the extract, and then use a filtrating paper piece, preferably a microfiltration membrane to remove the activated carbon which has adsorbed the agrochemicals.

According to each of the above-mentioned methods, from the agrochemical-containing Siraitia Grosvenorii extract, the agrochemicals can be removed to prepare a Siraitia Grosvenorii extract which does not contain the agrochemicals (agrochemical-free Siraitia Grosvenorii extract).

In present invention, the “agrochemical-free Siraitia Grosvenorii extract” denotes a Siraitia Grosvenorii extract in which the amount (remaining concentration) of (each of) the agrochemical(s) (at least one selected from the group consisting of dimethomorph, triadimenol, tebuconazole, difenoconazole, metalaxyl, and metalaxyl M is less than 0.01 ppm, the amount being in terms of that of a dry product of the substance concerned. The value “0.01 ppm” is a detection limit value of each of these agrochemicals.

In the invention, the wording “from an agrochemical-containing Siraitia Grosvenorii extract, the agrochemical(s) is/are removed” denotes that from a Siraitia Grosvenorii extract containing at least one selected from the group of dimethomorph, triadimenol, tebuconazole, difenoconazole, metalaxyl, and metalaxyl M (agrochemical-containing Siraitia Grosvenorii extract), the agrochemical(s) is/are removed to set the amount (remaining concentration) of (each of) the agrochemical (s) contained in the Siraitia Grosvenorii extract into less than 0.01 ppm, the amount being in terms of that of a dry product of the substance concerned.

Each of the agrochemicals contained in the Siraitia Grosvenorii extract can be analyzed (qualitatively and quantitatively), for example, by liquid chromatography/mass spectrometry (LC/MS) described in item (C) given below.

### (C) Agrochemical Analysis

LC/MS apparatus: LCMS-2010 EV (manufactured by Shimadzu Corp.),

LC Column: Inertsil ODS-3 (manufactured by GL Sciences Inc.; 250 mm×4.6 mm I.D. 5 μm),

LC conditions:
injected amount: 30 μL,
column temperature: 40° C.,
mobile phase: 80% by volume of acetonitrile and 20% by volume of water, and
flow rate: 1.0 mL/minute,

MS conditions:
ionizing method: APCI method, positive mode,
nebulizer gas flow rate: 2.5 L/minute,
APCI interface temperature: 350° C.,
CDL temperature: 200° C., and
heat block temperature: 300° C.

The agrochemical-free Siraitia Grosvenorii extract prepared by the method of the present invention has not only the characteristic that the amount (remaining concentration) of (each of) the agrochemical(s) (at least one agrochemical selected from the group consisting of dimethomorph, triadimenol, tebuconazole, difenoconazole, metalaxyl, and metalaxyl M) contained in the extract is less than 0.01 ppm, the amount being in terms of that of a dry product of the substance concerned, but also at least one of the following characteristics (A), and (B) and (C), preferably two thereof, more preferably all thereof:

(A) The recovery ratio of the Siraitia Grosvenorii glycoside (s) (mogroside V, mogroside IV, 11-oxo-mogroside V, and/or siamenoside I) in the present invention is 70% or more.

(B) The total amount of the Siraitia Grosvenorii glycosides (mogroside V, mogroside IV, 11-oxo-mogroside V, and siamenoside I) contained in the agrochemical-free Siraitia Grosvenorii extract is at least 33% by weight per 1.00% by weight of the agrochemical-free Siraitia Grosvenorii extract.

As described in the section “BACKGROUND ART”, it is known that the following extract has sweetness qualities very close to those of sugar about all basic performance of sweetness, which are “remaining sweetness, over-rich sweetness, peculiar sweetness, astringency, stimulus, refreshing, mellowness, relish and bitterness”: a Siraitia Grosvenorii extract (highly pure Siraitia Grosvenorii glycosides) purified to set the total amount of Siraitia Grosvenorii glycosides that are mogroside V, mogroside IV, 11-oxo-mogroside V, and siamenoside I to 33% or more by weight of the total, that is, the agrochemical-free Siraitia Grosvenorii extract.

An agrochemical-free Siraitia Grosvenorii extract prepared by the present invention is also preferably an extract in which the total amount of the Siraitia Grosvenorii glycosides (mogroside V, mogroside IV, 11-oxo-mogroside V, and siamenoside I) is 33% or more by weight in 100% by weight of the extract. The percentage by weight or proportion of the Siraitia Grosvenorii glycosides included in 100% by weight of the agrochemical-free Siraitia Grosvenorii extract, the percentage or the proportion being referred to in the invention, is a value in terms of dry weight (dry weight converted value)

The method for setting the percentage by weight of the Siraitia Grosvenorii glycosides in the agrochemical-free Siraitia Grosvenorii extract into 33% or more by weight as described above may be a method of subjecting, in advance, an agrochemical-containing Siraitia Grosvenorii extract not subjected to activated carbon treatment to one or more conventional purifying treatments, such as extraction, filtration, concentration, separation and drying, to remove components other than the Siraitia Grosvenorii glycosides, thereby adjusting the percentage by weight of the Siraitia Grosvenorii glycosides included in the Siraitia Grosvenorii extract to a large value, preferably 33% or more by weight.

The (total) percentage by weight of the Siraitia Grosvenorii glycosides (mogroside V, mogroside IV, 11-oxo-mogroside V, and siamenoside I) included in 100% by weight of the agrochemical-free Siraitia Grosvenorii extract can be gained by an HPLC analysis described below. Specifically, through the HPLC analysis, the peak area of each of the Siraitia Grosvenorii glycosides included in the agrochemical-free Siraitia Grosvenorii extract is compared with the peak area of the Siraitia Grosvenorii glycoside having an already known concentration (standard substance), whereby the quantity of the Siraitia Grosvenorii glycoside included in the agrochemical-free Siraitia Grosvenorii extract can be gained.

(C) The peak area which is detected by HPLC analysis of the agrochemical-free Siraitia Grosvenorii extract and is present near a retention period of 16 minutes is 3% or less of the total area of all peaks detected thereby.

As will be demonstrated by Experimental Example 3 given later, an agrochemical-free Siraitia Grosvenorii extract prepared by the method of the present invention has a characteristic that when the extract is subjected to HPLC under conditions described below, the area of a peak which is detected by the HPLC analysis and is present near a retention period of 16 minutes (hereinafter, the peak being to be referred to as “P₁₆”) is 3% or less of the total area of all peaks detected thereby.

### [HPLC Conditions]

Column: Kaseisorb LC ODS2000 (150 mm×4.6 mm I.D.) (manufactured by Tokyo Chemical Industry Co., Ltd.)

mobile phase: a mixed liquid of acetonitrile and water,
0 minutes→10 minutes (isocratic): 20% by volume of acetonitrile,
10 minutes→50 minutes (linear gradient): 20% by volume of acetonitrile→55% by volume thereof,

flow rate: 1 mL/minute,

column temperature: 40° C., and

detector: UV 203 nm.

In conclusion, the method of the present invention makes it possible to remove and decrease compounds detected as the peak (P₁₆ components) significantly to give an agrochemical-free Siraitia Grosvenorii extract in which the amount of the Pi components is significantly removed and decreased. As will be demonstrated in Experimental Example 4, the P₁₆ components are compounds each having a molecular weight of 358 daltons. Also as will be demonstrated in Experimental Example 4, the P₁₆ components are compounds related deeply to a decline in sweetness qualities (which results in off-favors) of a Siraitia Grosvenorii extract. Accordingly, the method of the invention makes it possible to remove and decrease, effectively from an agrochemical-containing Siraitia Grosvenorii extract, not only residual agrochemicals but also the compounds related deeply to a decline in sweetness qualities (which results in off-favors).

### (II) Agrochemical-Free Siraitia Grosvenorii Extract

The Siraitia Grosvenorii extract which is a subject matter of the present invention is a Siraitia Grosvenorii extract which does not contain agrochemicals (agrochemical-free Siraitia Grosvenorii extract) and which has the following characteristics (B) and (C):

(B) The total amount of mogroside V, mogroside IV, 11l-oxo-mogroside V, and siamenoside I included in the agrochemical-free Siraitia Grosvenorii extract is at least 33% by weight per 100% by weight of the agrochemical-free Siraitia Grosvenorii extract.

(C) The area of a peak which is detected by an HPLC analysis of the extract under conditions described below and which is around a retention time of 16 minutes is 3% or less of the total area of all peaks detected thereby.

The wording “around a retention time of 16 minutes” denotes that the peak is present, for example, between a retention time of 15 minutes and a retention time of 17 minutes although the wording is not limited. The peak area can be gained by using, for example, an analysis software Lab Solution (manufactured by Shimadzu Corp.)

### [HPLC Conditions]

Column: Kaseisorb LC ODS2000 (150 mm×4.6 mm I.D.) (manufactured by Tokyo Chemical Industry Co., Ltd.)

mobile phase: a mixed liquid of acetonitrile and water,
0 minutes→10 minutes (isocratic): 20% by volume of acetonitrile,
10 minutes→50 minutes (linear gradient): 20% by volume of acetonitrile→55% by volume thereof,

flow rate: 1 mL/minute,

column temperature: 40° C., and

detector: UV 203 nm.

The characteristics (B) and (C) are as described in the item (I). The description in the item (I) is incorporated hereinto for reference.

Agrochemicals that are objects against the agrochemical-free Siraitia Grosvenorii extract, which is a subject matter of the present invention, are dimethomorph, triadimenol, tebuconazole, difenoconazole, metalaxyl, and metalaxyl M. In other words, the agrochemical-free Siraitia Grosvenorii extract of the invention does not contain at least one selected from the group consisting of dimethomorph, triadimenol, tebuconazole difenoconazole, metalaxyl, and metalaxyl M. The extract does not preferably contain at least one of, for example, three of triadimenol, tebuconazole and difenoconazole out of the six agrochemicals, or the extract does not preferably contain the three agrochemicals at all. Alternatively, the agrochemical-free Siraitia Grosvenorii extract of the invention does not contain dimethomorph, triadimenol, tebuconazole, difenoconazole, metalaxyl nor metalaxyl M as agrochemicals.

The wording “not contain” means that the amount (remaining concentration) of each of the agrochemicals (dimethomorph, triadimenol, tebuconazole, difenoconazole, metalaxyl, and metalaxyl M) contained in the agrochemical-free Siraitia Grosvenorii extract of the present invention is less than 0.01 ppm, the amount being in terms of that of a dry product of the substance concerned. In other words, the agrochemical-free Siraitia Grosvenorii extract of the invention is an extract in which the amount (remaining concentration) of each of these specific agrochemicals is less than 0.01 ppm, the amount being in terms of that of a dry product of the substance concerned.

The agrochemical-free Siraitia Grosvenorii extract of the present invention, which is not restricted, is more preferably an extract which does not contain at least one of agrochemicals listed below. The wording “not contain” herein means that the amount (remaining concentration) of each of the agrochemicals listed below is less than 0.01 ppm, either, the amount being in terms of that of a dry product of the substance concerned.

1,1-Dichloro-2,2-bis(4-ethylphenyl)ethane, 1-naphthaleneacetic acid, 2-(1-naphthyl)acetamide, 2,2-DPA (2,2-dichloropropionic acid), 2,4,5-T(2,4,5-trichlorophenoxyacetic acid) 2,4-D(2,4-dichlorophenoxyacetic acid), 2,4-DB(4-(2,4-dichlorophenoxy)butyric acid), 4-chloro-phenoxyacetic acid, BHC (benzenehexachoride), DBEDC (dodecylbenzenesulfonic acid/bisethylenediamine copper complex [II]), DCIP (2,6-dichloroindophenol), DDT (dichlorodiphenyltrichloroethane), EPN (ethyl-p-nitrophenylthiobenzenesulfonate), EPTC (S-ethyl dipropylthiocarbamate), MCPA (2-(4-chloro-2-methyl-phenoxy)acetic acid), MCPB (4-(2-methyl-4-chlorophenoxy) butyric acid), sec-butylamine, TCMTB ([(benzothiazol-2-yithio)methyl] thiocyanate), XHC (3,5-dimethylphenyl methylcarbamate), γ-BHC (γ-benzene hexachloride), ioxynil, acrinathrin, azaconazole, azafenidin, azamethiphos, acifluorfen, acibenzolar-S-methyl, azimsulfuron, ashram, azinphos-methyl, acequinocyl, acetamiprid, acetochlor, acephate, azoxystrobin, azocyclotin, cyhexatin, atrazine, anilazine, anilofos, abamectin, amitraz, amitrole, ametryn, alachlor, alanycarb, aramite, aldicarb, aldoxycarb, aldrin, dieldrin, iodosulfuron methyl, isazophos, isouron, isocarbophos, isoxandifen-ethyl, isoxathion, isoxaflutole, isofenphos, isoprocarb, isoprothiolane, inabenfide, iprodione, iprovalicarb, iprobenfos, imazaquin, imazamethabenz-methyl ester, imazalil, imazosulfuron, imicyafos, imidacloprid, iminoctadine, imibenconazole, indanofan, indoxacarb, uniconazole P, esprocarb, ethametsulfuron-methyl, ethalfluralin, ethiofencarb, ethion, ethychlozate, ethiprole, edifenphos, ethephon, etoxazole, ethoxysulfuron, ethofenprox, ethofumesate, ethoprophos, etobenzanid, etridiazole, etrimfos, epoxiconazole, emamectin benzoate, endosulfan, endrin, oxadiazon, oxadixyl, oxazicomefon, oxabetrinil, oxamy, oxycarboxin, oxytetracycline, oxydemeton-methyl, oxyfluorfen, oxpoconazole fumarate, oxolinic acid, omethoate, orysastrobin, oryzalin, ortho-phenyl phenol, cadusafos, cafenstrole, captafol, cartap, thiocyclam, bensultap, carbaryl, carfentrazone ethyl, carpropamid, carbetamide, carbendazim, thiophanate, thiophanate-methyl, benomyl, carboxin, carbosulfan, carbofuran, quizalofop-ethyl, quinalphos, quinoxyfen, quinoclamine, quinomethionate, captan, quintozene, coumaphos, cumyluron, glyphosate, glufosinate, kresoxim-methyl, clethodim, cloquintocet mexyl, clodinafop propargyl, clodinafop acid, chlozolinate, clothianidin, clopyralid, clofencet, clofentezine, cloprop, clomazone, chromafenozide, clomeprop, clcransulam-methyl, chiorantraniiprole, chloridazon, chlorimuron-ethyl, chloroethoxfos, chlorsulfuron, chiothal dimethyl, chlordane, chlorpyrifos, chlorpyrifos-methyl, chlorfenapyr, chlorfenson, chlorfenvinphos, chlorbufam, chlorfluazuron, chlorpropharm, chlorbenside, chiormequat, chnloroxuron, chlorothaonil, chloroneb, chlorobenzilate, cyazofamid, cyanazine, cyanophos, diafenthiuron, diuron, diethofencarb, shienopirafen, dioxathion, dicamba, cyclanilide, cycloate, cycloxydim, diclocymet, diciosulam, cyclosulfamuron, dicrotophos, dichlorofenthion, dichlofluanid, cycloprothrin, dichlobenil, dichlofop-methyl, dichomezine, dicioran, dichilorprop, dichlorvos, naled, diquat, dicofol, disulfoton, dithianon, dithiopyr, diniconazole, cinidon-ethyl, dinocap, cinosulfuron, dinotefuran, cyhalothrin, cyhalofop butyl, dihydrostreptomycin, streptomycin, diphenamid, diphenyl, diphenylamine, difenoconazole, difenzoquat, cyfluthrin, cyflufenamid, diflufenican, diflubenzuron, cyproconazole, cyprodinil, cypermethrin, gibberellin, simazine, simeconazole, dimethametryn, dimethipin, dimethirimol, dimethylvinphos, dimethenamid, dimethoate, dimrethomorph, simetryn, dimepiperate, cymoxanil, silafluofen, cyromazine, cinmethylin, spinosad, spiroxamine, spirodiclofen, sulfentrazone, sulprophos, sulfosulfuron, sulfotep, sethoxydim, zoxamide, terbacil, diazinon, diallate, daimuron, dazomet, metam, methyl isothiocyanate, daminozide, thiacloprid, thiazinyl, thiazopyr, thiabendazole, thiamethoxam, thiodicarb, methomyl, thiobencarb, thiometon, thidiazuron, thifensulfuron methyl, thifluzamide, tecnazene, desmedipham, tetrachlorvinphos, tetraconazole, tetradifon, thenylchlor, tebuconazole, tebuthiuron, tebupirimfos, tebufenozide, tebufernpyrad, tepraloxydim, tefluthrin, teflubenzuron, demeton-S-methyl, deltamethrin, tralomethrin, terbutryn, terbufos, copper terephthalate, tralkoxydim, triadimenol, triadimefon, triasulfuron, triazophos, triallate, trichamide, triclopyr, trichlorfon, tricyclazole, triticonazole, tridemorph, trinexapac-ethyl, tribufos, triflusulfuronmethyl, triflumizole, triflumuron, trifluralin, trifloxystrobin, trifloxysulfuron, tribenulon-methyl, tolylfluanid, tolclofos-methyl, tolfenpyrad, naputaramu, naproanilide, napropamide, nicosulfuron, nicotine, nitenpyram, nitrapyrin, nitrothal-isopropyl, novaluron, norflurazon, baban, paclobutrazol, vamidothion, paraquat, parathion, parathion methyl, validamycin, halfenprox, haloxyfop, halosulfuron methyl, bioresmethrin, picolinafen, bispyribac sodium salt, bitertanol, bifenazate, bifenox, bifenthrin, piperonyl butoxide, piperophos, hymexazol, pymetrozine, pyraclostrobin, pyraclonil, pyraclofos, pyrazoxyfen, pyrazosulfuron ethyl, pyrazophos, pirazolynate, pyraflufen ethyl, pyridaphenthion, pyridaben, pyridalyl, pyridate, pyrifenox, pyriftalid, pyributicarb, pyriproxyfen, pirimicarb, pyrimidifen, pyriminobac-methyl, pirimiphos-methyl, pyrimethanil, pyrethrins, pyroquilon, vinclozolin, famufuru, famoxadone, fipronil, fenamiphos, fenarimol, fenitrothion, fenoxanil, fenoxaprop-ethyl, fenoxycarb, fenothiocarb, phenothrin, fenobucarb, ferimzone, fenamidone, fenchlorphos, fensulfothion, fenthion, fentin, phenthoate, fentrazamide, fenvalerate, fenpyroximate, fenbuconazole, fenpropathrin, fenpropimorph, fenhexamid, phenmedipham, fthalide, butachlor, butafenacil, butamifos, butyrate, butroxydim, bupirimate, buprofezin, flazasulfuron, furathiocarb, primisulfuron-methyl, furametpyr, primisulfuron methyl, furilazole, fluacrypyrim, fluazinam, fluazifop, fluopicolide, fluometuron, fluquinconazole, fludioxonil, flucythrinate, flusilazole, flusulfamide, fluthiacet methyl, flutolanil, flutriafol, fluvalinate, flufenacet, flufenoxuron, flufenpyr-ethyl, flubendiamide, flamioxazin, flumiciorac pentyl, flumetsulam, fluridone, fluroxypyr, pretilachlor, prochloraz, procymidone, prosulfuron, prothiofos, flonicamid, propquizafop, propachlor, propazine, propanil, propaphos, propamocarb, propargite, propiconazole, propyzamide, prohydrojasmon, propham, profenofos, prohexadione calcium salt, propetamphos, propoxyphene carbazone, propoxur, bromacil, prometryn, bromoxynil, bromobutide, bromopropylate, bromophos, bromophos-ethyl, florasulam, hexachlorobenzene, hexaconazole, hexazinone, hexaflumuron, hexythiazox, benalaxyl, benoxacor, penoxsulam, heptachlor, permethrin, penconazole, pencycuron, bensulide, bensulfuron methyl, benzobicyclon, benzofenap, bendiocarb, bentazone, benthiavalicarb-isopropyl, pendimethalin, pentoxazone, benfuracarb, benfluralin, benfuresate, phoxim, phosalone, boscalid, fosthiazate, phosphamidon, phosmet, fosetyl, fomesafen, foramsulfuron, forchlorfenuron, folpet, formothion, phorate, malathion, maleic hydrazide, mandipropamid, myclobutanil, milbemectin, mecarbam, mnecoprop, metsulfluro methyl, metaldehyde, methacrifos, methabenzthiazuron, methamidophos, metamitron, the sum of metalaxyl and mefenoxam, methiocarb, methidathion, methoxychlor, methoxyfenozide, metconazole, metosulam, metsulfuron methyl, methoprene, metominostrobin, metolachlor, metribuzin, mepanipyrim, mepiquat chloride, mevinphos, mefenacet, mefenpyr-diethyl, mepronil, monocrotophos, mononolinron, molinate, lactofen, linuron, rimsulfuron, lufenuron, resmethrin, lenacil, fenbutatin oxide, propylene oxide and ethylene dibromide.

The agrochemical-free Siraitia Grosvenorii extract is preparable by using, as a raw material, an agrochemical-containing Siraitia Grosvenorii extract having at least one selected from dimethomorph, triadimenol, tebuconazole, difenoconazole, metalaxyl, and metalaxyl M to remove the agrochemical (s) by the preparing method according to the above-mentioned item (I).

The agrochemical-free Siraitia Grosvenorii extract of the present invention is high in safety since the extract does not contain at least the agrochemicals (dimethomorph, triadimenol, tebuconazole, difenoconazole, metalaxyl, and metalaxyl M). Moreover, the extract has the characteristics (B) and (C) not to be lowered in sweetness qualities (or not to have off-flavors), so that the extract has good sweetness qualities about eight basic items (remaining flavor, over-rich flavor, peculiar flavor, astringency, stimulating flavor, refreshing flavor, mellowness, relish, and bitterness) that are basic performances of sweetness so as to be useful as a sweetener composition having good sweetness qualities close to those of sugar.

### (III) Sweetener Composition and Product Including the Same

The sweetener composition of the present invention is a composition including the above-mentioned agrochemical-free Siraitia Grosvenorii extract, or a composition including not only the agrochemical-free Siraitia Grosvenorii extract but also an auxiliary sweetener constituent.

The auxiliary sweetener constituent means a constituent having sweetness, or a constituent for adjusting the sweet intensity of a sweetener and a sweetness quality thereof. Examples thereof include saccharide sweeteners such as monosaccharides, disaccharides, oligosaccharides, and sugar alcohols; non-saccharide sweeteners; and dietary fibers.

Examples of the monosaccharides among the saccharide sweeteners include fructose, glucose, xylose, sorbose, galactose, and isomerized sugar. Examples of the disaccharides include maltose, lactose, trehalose, sucrose, isomerized lactose and palatinose. Examples of the oligosaccharide, xylooligosaccharide, fructooligosaccharride, soybean oligosaccharide, isomaltooligosaccharide, lactosucrose, galactooligosaccharide, lactulose, palatinose oligosaccharides, sucro-oligosaccharide, and theanine-oligosaccharide and seaweed oligosaccharides. Examples of the sugar alcohols included maltitol, xylitol, sorbitol, mannitol, and palatinit. Examples of synthetic sweeteners among the non-saccharide sweeteners include aspartame, advantame, acesulfame K, sucralose, and neotame.

Examples of natural sweeteners among the non-saccharide sweeteners include licorice extract, stevia extract, and thaumatin extract.

Examples of the dietary fibers include pectin, guar bean enzymatic decomposition product, glucomannan, β-glucan, polydextrose, inulin, agarose, sodium alginate, carrageenan, fucoidan, porphyran, laminaran, and water-soluble dietary fibers such as indigestible dextrin; and insoluble dietary fibers such as cellulose, hemicellulose, lignin, chitin, and chitosan. Preferred are water-soluble dietary fibers.

When the sweetener composition of the present invention is a composition including not only the agrochemical-free Siraitia Grosvenorii extract but also an auxiliary sweetener constituent, the proportion of the agrochemical-free Siraitia Grosvenorii extract included in the sweetener composition may be from 0.01 to 99.99% by weight, preferably from 0.1 to 99.9% by weight in 100% by weight of the composition. The proportion of the auxiliary sweetener constituent may be from 0.01 to 99.99% by weight, preferably from 0.1 to 99.9% by weight of the composition.

As described above, the sweetener composition of the present invention is a composition including the agrochemical-free Siraitia Grosvenorii extract of the invention, or a composition including not only the agrochemical-free Siraitia Grosvenorii extract but also an auxiliary sweetener constituent, so as to be high in safety. Additionally, the composition is not lowered in sweetness qualities (or does not give off-flavors), and has good sweetness qualities close to those of sugar. Thus, the composition is favorably usable as a sweetener for food and drink, quasi-drugs, medicines or cosmetic products. Examples of the cosmetic product include lipsticks, lip cream, and other cosmetic articles each used for lips.

When the sweetener composition of the present invention is made of the agrochemical-free Siraitia Grosvenorii extract of the invention, the sweetness degree thereof is from 200 to 400 times that of sugar (sucrose). This sweetness degree is appropriately adjustable by blending the auxiliary sweetener constituent into the agrochemical-free Siraitia Grosvenorii extract.

A Siraitia Grosvenorii extract prepared by the method of the present invention, as well as Siraitia Grosvenorii extract of the invention and the sweetener composition of the invention, makes it possible to provide more safely a low-energy (low-calorie) or zero-energy (zero-calorie) sweetener composition. This low-energy or zero-energy sweetener composition is useful as a sweetener composition for energy-taking-in restricted persons such as obesity patients and diabetics, or for dieting-required or -desired persons.

### (IV) Off-Flavor-Removed Siraitia Grosvenorii Extract

A Siraitia Grosvenorii extract prepared by the method of the present invention, or the Siraitia Grosvenorii extract of the invention makes it possible to recover, with a high yield, mogrosides (Siraitia Grosvenorii glycosides) that are mogroside V, mogroside IV, 11-oxo-mogroside V and siamenoside I, remove agrochemicals selectively and efficiently, and further remove and decrease specific off-flavor components contained in the Siraitia Grosvenorii extract. Thus, the present invention can give Siraitia Grosvenorii extracts having better sweetness qualities than Siraitia Grosvenorii extracts prepared by any conventional method.

Accordingly, the activated-carbon-used method of the present invention is also applicable to Siraitia Grosvenorii yielded using, for example, a cultivating method which uses no agrochemical or hardly uses an agrochemical. Specifically, the activated-carbon-used method of the invention is added into a process performed when a Siraitia Grosvenorii extract is extracted, or a Siraitia Grosvenorii extract which has been extracted once by a conventional method is further subjected to the activated carbon treatment, whereby the specific off-flavor components can be removed and decreased. Thus, in any case, the invention can give Siraitia Grosvenorii extracts having better sweetness qualities than Siraitia Grosvenorii extracts prepared by any conventional method.

### EXAMPLES

Hereinafter, the present invention will be described in more detail by way of experimental examples, working examples, comparative examples and others. However, the experimental examples and the others are intended to be demonstrated as examples, so as not to restrict the scope of the invention. Other aspects, advantages and modifications within the scope of the invention are evident for those skilled in the art who are involved in fields related to the invention.

### Reference Example 1: Quantitative Method of Siraitia Grosvenorii Glycosides

Mogroside V and other Siraitia Grosvenorii glycosides (mogroside IV, 11-oxo-mogroside V, and siamenoside I) included in a Siraitia Grosvenorii extract were quantitatively determined, using high performance liquid chromatography (HPLC). An apparatus and conditions used therein are as follows:

### (1) Apparatus and Conditions Used for Quantitative Determination of Mogroside V

HPLC apparatus: Prominence LC-20AD (manufactured by Shimadzu Corp.),

LC Column: Shodex Asahipak NI-2-50 4E (manufactured by Show Denko K.K.; 250 mm×4.6 mm I.D.), and

LC conditions:
injected amount: 20 μL,
column temperature: 40° C.,
mobile phase: 74% by volume of acetonitrile and 26% by volume of water,
flow rate: 1.0 mL/minute, and
detecting wavelength: UV 203 nm.

### (2) Apparatus and Conditions Used for Quantitative Determination of Other Siraitia Grosvenorii Glycosides

HPLC apparatus: Prominence LC-20AD (manufactured by Shimadzu Corp.),

LC column: Kaseisorb LC ODS 2000 (manufactured by Tokyo Chemical Industry Co., Ltd.; 150 mm×4.6 mm I.D.), and

LC conditions:
injected amount: 20 μL,
column temperature: 40° C.,
mobile phase: phase A: water, and phase B: acetonitrile,
0 minutes→10 minutes (isocratic): 80% by volume of phase A, and 20% by volume of phase B,
10 minutes→50 minutes (linear gradient): “80% by volume of phase A, and 20% by volume of phase B”→“45% by volume of phase A, and 55% by volume of phase B”,
flow rate: 1.0 ml/minute, and
detecting wavelength: UV 203 nm.

### Reference Example 2: Agrochemical Content Measurement

### (1) Pretreatment

Before agrochemicals contained in a Siraitia Grosvenorii extract are quantitatively determined by liquid chromatography/mass spectrometry (LC/M S), the Siraitia Grosvenorii extract is pretreated by steps described below. The resultant treated product was used as an “LS/MS measuring sample”.

Into a 200 ml volume measuring-flask is weighed 2 g of the Siraitia Grosvenorii extract, and water is then added thereto. Thereafter, water further added thereto to prepare a 200 mL constant volume, and this is used as a “constant volume liquid 1”. A 20 mL volume transfer pipet is used to fractionize a volume of 20 mL from the constant volume liquid 1. This is added to a 500 mL volume separating funnel in which 200 mL of water containing 10 g of sodium chloride is charged. Thereto is added 50 mL of ethyl acetate/n-hexane (1:3). The resultant is shaken for 1 minute and then allowed to stand still for about 30 minutes. Therefrom, the organic phase is separated and collected. This operation is further repeated two times, and the resultant organic phases are combined with each other. To the collected organic phase is added 20 g of sodium sulfate, and the resultant system is allowed to stand still for 30 minutes or longer to dry the organic phase. From the dried organic phase, a filtrating paper piece (5A, manufactured by Advantec Toyo Kaisha, Ltd.; 125 mm) is used to filtrate away sodium sulfate, and then the filtrate is then collected. The used compound sodium sulfate and filtrating paper piece are washed with 20 mL of ethyl acetate/n-hexane (1:3), and the wash liquid is combined with the filtrate. This filtrate is concentrated and dried into a solid form, using a rotary evaporator. The concentrated product is dissolved into a small volume of acetone, and the solution is shifted into a 50 mL eggplant shaped flask. The solution is then concentrated and dried into a solid form. This dried product is dissolved into methanol, and the solution is shifted into a 2 mL volume measuring-flask. Furthermore, the 50 mL volume eggplant shaped flask is washed with a small volume of methanol, and the wash liquid is shifted into the above-mentioned 2 mL volume measuring-flask. Methanol is then added thereto to prepare a 2 mL constant volume. This is used as a “constant volume liquid 2”. This constant volume liquid 2 is filtrated through a product EKICRODISC 3 (manufactured by NIPPON Genetics Co, Ltd.), and the filtrate is collected. After a while, the prepared filtrated is used as an “LS/MS measuring sample”.

### (2) Quantitative Determination of Agrochemicals Contained in Siraitia Grosvenorii Extract

Agrochemicals (dimethomorph, triadimenol, tebuconazole, difenoconazole, metalaxyl, and metalaxyl M) contained in the Siraitia Grosvenorii extract are quantitatively determined by liquid chromatography/mass spectrometer (LC/MS). An apparatus and conditions used therein were as described below. The quantitative determination limit of each of the agrochemicals according to this method is 0.01 ppm.

### (2-1.) Apparatus and Conditions Used in Quantitative Determination of Agrochemicals

LC/MS apparatus: LCMS-2010 EV (manufactured by Shimadzu Corp.),

LC Column: Inertsil ODS-3 (manufactured by GL Sciences Inc.; 250 mm×4.6 mm I.D. 5 μm),

LC conditions:
injected amount: 30 μL,
column temperature: 40° C.,
mobile phase: 80% by volume of acetonitrile and 20% by volume of water, and
flow rate: 1.0 mL/minute,

MS conditions:
ionizing method: APCI method, positive mode,
nebulizer gas flow rate: 2.5 L/minute,
APCI interface temperature: 350° C.,
CDL temperature: 200° C., and
heat block temperature: 300° C.

### (2-2) Method for Calculating Out Agrochemical Removal Ratio

The removal ratio of each of the agrochemicals according to the agrochemical removing treatment can be gained in accordance with the following expression (1):

[Math. 4]

Agrochemical removal ratio (%)={A₀−A₁)/A₀}×100(%) (1)

A₀: Agrochemical content (ppm) in Siraitia Grosvenorii extract before agrochemical treatment
A₁: Agrochemical content (ppm) in Siraitia Grosvenorii extract after agrochemical treatment

### Reference Example 3: Proportion of Each of Agrochemicals and Siraitia Grosvenorii Glycosides Contained in Raw Material Siraitia Grosvenorii Extract

Each of the following was gained by the methods descried in Reference Examples 1 and 2 described above: the content of Siraitia Grosvenorii glycosides (mogroside V, mogroside IV, 11-oxo-mogroside V, and siamenoside I) included in a Siraitia Grosvenorii extract (Luchanguo glucosides 40% yellow type) available from Guilin Rheinland Biological Technology Co., Ltd. (hereinafter, this extract will be referred to also as the “raw material Siraitia Grosvenorii extract”); and the content of agrochemicals (dimethomorph, triadimenol, tebuconazole, difenoconazole, metalaxyl, and metalaxyl M) included in the same.

The results are shown in Tables 3 and 4. Each value shown in each of the tables is a dried-product-amount-converted value of each of the components contained in the raw material Siraitia Grosvenorii extract (dried product)

TABLE 3

| | Siraitia Grosvenorii glycoside content (%) | | | |
|---|---|---|---|---|
| | Mogroside | Mogroside | 11-Oxo- | |
| | V | IV | mogroside V | Siamenoside I |
| Raw material | 45.0 | 2.8 | 6.7 | 2.0 |
| Siraitia | | | | |
| Grosvenorii | | | | |
| extract | | | | |

TABLE 4

| | Agrochemical content (ppm) | | | | |
|---|---|---|---|---|---|
| | | | | | Metalaxyl |
| | | | | | and |
| | | | | | metalaxyl M |
| | Dimethomorph | Tiadimenol | Tebuconazole | Difenoconazole | (total sum) |
| Raw material | 0.11 | 0.39 | 0.02 | 0.02 | 0.03 |
| Siraitia | | | | | |
| Grosvenorii | | | | | |
| extract | | | | | |

### Experimental Example 1: Preparation of Agrochemical-Free Siraitia Grosvenorii Extract, Using Activated Carbon

(1) Into an aqueous solution containing 25% by volume of ethanol was dissolved 5 g of the raw material Siraitia Grosvenorii extract (Luohanguo glucosides 40% yellow type) available from Guilin Rheinland Biological Technology Co., Ltd. to prepare a 500 mL constant volume. A pump was used to inject, at a flow rate of 3.3 mL/minute, this constant volume liquid into a column (column inside diameter: 15 mm; and length: 300 mm) filled with 40 mL of a (granular) activated carbon (in each of Example 1, and Comparative Examples 1 to 3) swollen, one night or longer in advance, with 25% by volume of ethanol and 75% by volume of water.

### [Each Activated Carbon]

### Example 1

iodine adsorption ability=1610 mg/g (FG-3: manufactured by Japan Enviro Chemicals, Ltd.),

### Comparative Example 1

iodine adsorption ability=990 mg/g (BROCOAL CM, manufactured by Taihei Chemical Industrial Co., Ltd.),

### Comparative Example 2

iodine adsorption ability 1000 mg/g and <1500 mg/g (KURARAY COAL: manufactured by Kuraray Chemical Co., Ltd.), and

### Comparative Example 3

iodine adsorption ability ≥1000 mg/g and <1500 mg/g (GCN 1240 PULS: manufactured by Cabot Norit Japan Co., Ltd.)

About the swollen activated carbons each having the volume of 40 mL, the respective weights of the products FP-3, BROCOAL CM, KURAPAY COAL KL, and GCN 1240 PULS were 13.84 g, 17.46 g, 18.04 g, and 20.67 g, respectively.

(2) The entire amount (500 mL) of the Siraitia Grosvenorii extract solution was injected into the column to collect the eluted liquid, and then 200 mL of 25% by volume of ethanol and 75% by volume of water was injected thereinto at a flow rate of 3.3 mL/minute to wash the activated carbon. The wash liquid was then collected. The collected eluted liquid and wash liquid were mixed with each other. The resultant collected liquid was concentrated under a reduced pressure to remove ethanol. A freeze-drying machine VD-800R (Tietech Co., Ltd.) was then used to yield a granular activated carbon treated product.

These operations were each made at room temperature. [010 7]

(3) About each of the activated carbon treated products (0.2% by weight/weight aqueous solution) that was yielded in the item (2), the amount of each of the Siraitia Grosvenorii glycosides (mogroside V, mogroside IV, 11-oxo-mogroside V, and siamenoside I) included in the activated carbon treated product (each of Example 1, and Comparative Examples 1 to 3) was measured in accordance with the method described in Reference Example 1. Moreover, in accordance with the method described in Reference Example 2, the item (1), the pretreatment was made, and then in accordance with the method in the item (2) a measurement was made about the amount of each of the agrochemicals (dimethomorph, triadimenol, tebuconazole, difenoconazole, metalaxyl, and metalaxyl M) contained in the activated carbon treated product (each of Example 1, and Comparative Examples 1 to 3).

Next, from each of the measured contents, the Siraitia Grosvenorii glycoside recovery ratio (%) and the remaining agrochemical removal ratio (%) according to the treatment with each of the activated carbons were calculated. These values are shown in Tables 5 and 6.

TABLE 5

| | | Siraitia Grosvenorii glycoside | | |
|---|---|---|---|---|
| | | recovery ratio (%) | | |
| | Activated carbon | | 11-Oxo- | Mogroside IV |
| | (iodine adsorption | Mogroside | mogroside | (containing |
| | ability: mg/g) | V | V | siamenoside) |
| Example 1 | FG-3 | 76 | 68 | 78 |
| | (1610) | | | |
| Comparative | BROCOAL CM | 92 | 98 | 88 |
| Example 1 | (990) | | | |
| Comparative | KURARAY | 97 | 100 | 93 |
| Example 2 | COAL KL | | | |
| | (≥1000) | | | |
| Comparative | GCN 1240 | 90 | 76 | 89 |
| Example 3 | PLUS | | | |
| | (>1000) | | | |

TABLE 6

| | Activated | | | | | |
|---|---|---|---|---|---|---|
| | carbon | | | | | |
| | (iodine | Remaining agrochemical removal ratio (%) | | | | |
| | adsorption | | | | | Metalaxyl |
| | ability: | | | | | and |
| | mg/g) | Dimethomorph | Triadimenol | Tebuconazole | Difenoconazole | metalaxyl M |
| Example 1 | FG-3 | 100 | 100 | 100 | 100 | 100 |
| | (1610) | | | | | |
| Comparative | BROCOAL CM | 0 | 43 | 0 | 100 | 0 |
| Example 1 | (990) | | | | | |
| Comparative | KURARAY COAL | 0 | 83 | 0 | 0 | 49 |
| Example 2 | KL | | | | | |
| | (≥1000) | | | | | |
| Comparative | GCN 1240 | 0 | 55 | 0 | 100 | 24 |
| Example 3 | PLUS | | | | | |
| | (>1000) | | | | | |

The amount of each of the agrochemicals (dimethomorph, triadimenol, tebuconazole, difenoconazole, metalaxyl, and metalaxyl M) contained in the activated carbon treated product in Example 1 was less than the detection limit amount (less than 0.002 ppm) thereof. It was verified that these agrochemicals were each removed.

As understood from the results, it has been verified that the use of an activated carbon having an iodine adsorption ability of at least 1500 mg/g or more, preferably 1600 mg/g or more makes it possible to remove 100% of residual agrochemicals (dimethomorph, triadimenol, tebuconazole, difenoconazole, metalaxyl, and metalaxyl M) contained in a Siraitia Grosvenorii extract. The distribution coefficient (LogPow) of each of these residual agrochemicals (dimethomorph, triadimenol, tebuconazole, difenoconazole, metalaxyl, and metalaxyl M) was 1.71 or more. This matter would demonstrate that a compound having a distribution coefficient (LogPow) of at least 1.71 or more can be removed by treatment with an activated carbon having an iodine adsorption ability of 1500 mg/g or more, preferably 1600 mg/g or more.

It has also been verified that the treatment with the activated carbon makes it possible to recover, with a yield of 70% or more, the Siraitia Grosvenorii glycosides (mogroside V, mogroside IV, 11-oxo-mogroside V, and siamenoside I) included in an agrochemical-containing Siraitia Grosvenorii extract before the activated carbon treatment thereof. It has also been verified that the total amount of the Siraitia Grosvenorii glycosides (mogroside V, mogroside IV, 11-oxo-mogroside V, and siamenoside I) included in the agrochemical-free Siraitia Grosvenorii extract after the activated carbon treatment is 33% or more by weight per 100% by weight of the agrochemical-free Siraitia Grosvenorii extract.

### Experimental Example 2: Preparation of Agrochemical-Free Siraitia Grosvenorii Extract, Using Each Activated Carbon

(1) into water was dissolved 5 g of the raw material Siraitia Grosvenorii extract (Luohanguo glucosides 40% yellow type, available from Guilin Rheinland Biological Technology Co., Ltd.) to prepare a 50 mL constant volume. The whole of this constant volume was shifted into a baker, and to this liquid was added 1 g of the (powdery) activated carbon (each of Examples 2 to 4, and Comparative Examples 4 to 7). A stirrer was used to stir this liquid for 30 minutes, and then a 0.45-μm membrane filter was used to filtrate the stirred liquid under a reduced pressure to remove the activated carbon. The resultant filtrate was freeze-dried to yield an activated carbon treated product. These operations were each made at room temperature.

### [Each Powdery Activated Carbon]

### Example 2

caramel decolorizing ability 86.2% (KURARAY COAL PK-D, manufactured by Kuraray Chemical Co., Ltd.), and particle size: 75 to 86 μm both inclusive,

### Example 3

caramel decolorizing ability 94.6% (FP-1, manufactured by Japan Enviro Chemicals, Ltd.),

### Example 4

caramel decolorizing ability 95.7% (FP-6, manufactured by Japan Enviro Chemicals, Ltd.),

### Comparative Example 4

caramel decolorizing ability 73.1% (FP-4, manufactured by Japan Enviro Chemicals, Ltd.),

### Comparative Example 5

caramel decolorizing ability 17.6% (FP-8, manufactured by Japan Enviro Chemicals, Ltd.),

### Comparative Example 6

caramel decolorizing ability 70.9% (FP-4, manufactured by Japan Enviro Chemicals, Ltd.), and

### Comparative Example 7

caramel decolorizing ability 80% (BROCOAL B mark active carbon, manufactured by Taihei Chemical Industrial Co., Ltd.)

(2) About each of the activated carbon treated products that was yielded in the item (1), in accordance with the method described in Reference Example 1, the amount of each of the Siraitia Grosvenorii glycosides (mogroside V, mogroside IV, 11-oxo-mogroside V, and siamenoside I) included in the activated carbon (in each of Examples 2 to 4, and Comparative Examples 4 to 7) was measured. Moreover, in accordance with the method described in Reference Example 2, the item (1), the pretreatment was made, and in accordance with the method in the item (2) a measurement was made about the amount of each of the agrochemicals (dimethomorph, triadimenol, tebuconazole, difenoconazole, metalaxyl, and metalaxyl M) contained in the activated carbon treated product (in each of Examples 2 to 4, and Comparative Examples 4 to 7). Next, from each of the measured contents, the Siraitia Grosvenorii glycoside recovery ratio (%), the remaining agrochemical detected amount (ppm) and the remaining agrochemical removal ratio (%) according to the treatment with each of the activated carbons were calculated.

These values are shown in Tables 7 to 9.

TABLE 7

| | | Siraitia Grosvenorii glycoside | | |
|---|---|---|---|---|
| | Activated carbon | recovery ratio (%) | | |
| | (caramel | | 1,1-Oxo- | Mogroside IV |
| | decolorizing | Mogroside | mogroside | (containing |
| | ability) | V | V | siamenoside I) |
| Example 2 | PK-D | 88 | 87 | 98 |
| | (86.2%) | | | |
| Example 3 | FP-1 | 86 | 96 | 92 |
| | (94.6%) | | | |
| Example 4 | FP-6 | 89 | 91 | 90 |
| | (95.7%) | | | |
| Comparative | FP-4 | 93 | 97 | 98 |
| Example 4 | (73.1%) | | | |
| Comparative | FP-8 | 96 | 92 | 100 |
| Example 5 | (17.6%) | | | |
| Comparative | FP-9 | 94 | 95 | 100 |
| Example 6 | (70.9%) | | | |
| Comparative | BROCOAL B | 90 | 79 | 93 |
| Example 7 | mark | | | |
| | (80%) | | | |

TABLE 8

| | Activated | | | | | |
|---|---|---|---|---|---|---|
| | carbon | Remaining agrochemical detected amount (ppm) | | | | |
| | (caramel | | | | | Metalaxyl |
| | decolorizing | | | | | and |
| | ability) | Dimethomorph | Triadimenol | Tebuconazole | Difenoconazole | metalaxyl M |
| Example 2 | PK-D | N.D. | N.D. | N.D. | N.D. | N.D. |
| | (86.2%) | | | | | |
| Example 3 | FP-1 | N.D. | N.D. | N.D. | N.D. | N.D. |
| | (94.6%) | | | | | |
| Example 4 | FP-6 | N.D. | N.D. | N.D. | N.D. | N.D. |
| | (95.7%) | | | | | |
| Comparative | FP-4 | 0.039 | 0.201 | 0.009 | N.D. | 0.018 |
| Example 4 | (73.1%) | | | | | |
| Comparative | FP-8 | 0.110 | 0.381 | 0.020 | 0.019 | 0.030 |
| Example 5 | (17.6%) | | | | | |
| Comparative | FP-9 | 0.089 | 0.241 | 0.012 | N.D. | 0.018 |
| Example 6 | (70.9%) | | | | | |
| Comparative | BROCOAL B | 0.034 | 0.112 | N.D. | N.D. | 0.019 |
| Example 7 | mark | | | | | |
| | (80%) | | | | | |

TABLE 9

| | Activated | | | | | |
|---|---|---|---|---|---|---|
| | carbon | Remaining agrochemical removal ratio (%) | | | | |
| | (caramel | | | | | Metalaxyl |
| | decolorizing | | | | | and |
| | ability) | Dimethomorph | Triadimenol | Tebuconazole | Difenoconazole | metalaxyl M |
| Example 2 | PK-D | 100 | 100 | 100 | 100 | 100 |
| | (86.2%) | | | | | |
| Example 3 | FP-1 | 100 | 100 | 100 | 100 | 100 |
| | (94.6%) | | | | | |
| Example 4 | FP-6 | 100 | 100 | 100 | 100 | 100 |
| | (95.7%) | | | | | |
| Comparative | FP-4 | 65 | 48 | 55 | 100 | 40 |
| Example 4 | (73.1%) | | | | | |
| Comparative | FP-8 | 0 | 2 | 0 | 5 | 0 |
| Example 5 | (17.6%) | | | | | |
| Comparative | FP-9 | 19 | 38 | 40 | 100 | 40 |
| Example 6 | (70.9%) | | | | | |
| Comparative | BROCOAL B | 69 | 71 | 100 | 100 | 37 |
| Example 7 | mark | | | | | |
| | (80%) | | | | | |

As understood from the results, it has been verified that the use of an activated carbon having a caramel decolorizing ability of at least 85% makes it possible to remove 100% of residual agrochemicals (dimethomorph, triadimenol, tebuconazole, difenoconazole, metalaxyl, and metalaxyl M) contained in a Siraitia Grosvenorii extract (the amount of each of the agrochemicals: less than the detection limit [0.002 ppm] thereof). About the activated carbon treated product in Example 4, the remaining amount of each of the agrochemicals described in the paragraph [0078] of the document DESCRIPTION was measured. As a result, the amount was less than the detection limit [0.01 ppm] thereof. Thus, it was verified that each of these agrochemicals was not included in the product.

It has been verified that the treatment with the activated carbon makes it possible to recover, with a high yield of at least 85% by weight, preferably 90% or more by weight, the Siraitia Grosvenorii glycosides (mogroside V, mogroside IV, 11l-oxo-mogroside V, and siamenoside I) included in an agrochemical-containing Siraitia Grosvenorii extract before the activated carbon treatment thereof. This matter has made it evident that the use of an activated carbon having a caramel decolorizing ability of at least 85% makes it possible to remove residual agrochemicals selectively and efficiently while Siraitia Grosvenorii glycosides useful for sweetener constituents are recovered with a high yield.

It has also been verified that the treatment with the activated carbon makes it possible to recover, with a yield of 70% or more, specifically 80% or more, more specifically 85% or more, the Siraitia Grosvenorii glycosides (mogroside V, mogroside IV, 11-oxo-mogroside V, and siamenoside I) included in an agrochemical-containing Siraitia Grosvenorii extract before the activated carbon treatment thereof. It has also been verified that the total amount of the Siraitia Grosvenorii glycosides (mogroside V, mogroside IV, 11-oxo-mogroside V, and siamenoside I) included in the agrochemical-free Siraitia Grosvenorii extract after the activated carbon treatment is 33% or more by weight per 100% by weight of the agrochemical-free Siraitia Grosvenorii extract.

### Experimental Example 3: Component Analysis and Sweetness Quality Evaluation of Agrochemical-Free Siraitia Grosvenorii Extract

### (1) Component Analysis

The activated carbon untreated product (raw material Siraitia Grosvenorii extract) and the activated carbon treated product (in each of Examples 3 and 4 and Comparative Example 7) were supplied to high performance liquid chromatography with the HPLC apparatus and under conditions described in Reference Example 1, the item (2) (Quantitative Method of Siraitia Grosvenorii Glycosides) By making a comparison between the resultant chromatogram of the activated carbon untreated product and that of the activated carbon treated product, it was verified that the area of a peak detected around a retention time of 16 minutes (the area and the peak will be referred to also as “P₁₆” and “P₁₆ area”, respectively, hereinafter) was remarkably decreased by the activated carbon treatment.

Out of the activated carbon treated products, Examples 4 and 3 were significantly smaller in P₁₆ area than Comparative Example 7. Thus, it has been verified that treatment with an activated carbon having a caramel decolorizing ability of at least 85%, preferably 90% or more makes it possible to remove and decrease P_{F}, components effectively from a Siraitia Grosvenorii extract.

Table 8 shows, about each of the examples, the proportion (%) of the P₁₆ area in the total area of all peals detected by the HPLC under the above-mentioned conditions. As will be shown below, in each of the activated carbon treated products (in Examples 3 and 4), the proportion (%) of the P₁₆ area in the total area of all the peaks was 3% or less, specifically 2.5% or less.

About each of the areas, an apparatus “Lab Solutions” (manufactured by Shimadzu Corp.) was used to gain the area value of each of the peaks. The proportion (%) of each of the P₁₆ areas was calculated out as the ratio thereof to the total value of the respective areas of all the peaks. The wording “around a retention time of 16 minutes” denotes the proportion of peaks between retention times of 15 to 17 minutes in the area of all the peaks.

TABLE 10

| | | | | Ratio relative to |
|---|---|---|---|---|
| | | | Proportion | proportion (%) (described |
| | | Caramel | (%) of P₁₆ | leftward and regarded as |
| | | decolorizing | area in total | 100) of activated carbon |
| | | ability | peak area | untreated product |
| Activated carbon | | — | 9.97 | 100 |
| untreated product | | | | |
| Activated carbon | Example 3 | 94.6% | 2.38 | 23.87 |
| treated products: | | | | |
| | Example 4 | 95.7% | 1.46 | 14.64 |
| | Comparative | 80.0% | 4.13 | 41.42 |
| | Example 7 | | | |

The Siraitia Grosvenorii extract (activated carbon treated product: Example 1) treated with the activated carbon, the iodine adsorption ability of which was 1500 mg/g or more, in Experimental Example 1 was also subjected to the same activated carbon treatment. As a result, it was verified that from the raw material Siraitia Grosvenorii extract, the P₁₆ components were removed and decreased.

TABLE 11

| | | | | Ratio relative to |
|---|---|---|---|---|
| | | | Proportion | proportion (%) (described |
| | | Iodine | (%) of P₁₆ | leftward and regarded as |
| | | adsorption | area in total | 100) of activated carbon |
| | | ability | peak area | untreated product |
| Activated carbon | | — | 13.39 | 100 |
| untreated product | | | | |
| Activated carbon | Example 1 | 1610 | 2.38 | 7.02 |
| treated products: | | | | |
| | Comparative | 990 | 5.29 | 39.51 |
| | Example 1 | | | |
| | Comparative | ≥1000 | 1.86 | 13.89 |
| | Example 2 | | | |
| | Comparative | >1000 | 6.37 | 47.57 |
| | Example 3 | | | |

### (2) P₁₆ Component Identification

The P₁₆ components were separated and collected, and then supplied to an C/MS analysis under the conditions described in Reference Example 2, the item (2), (2-1). As a result, it was verified that the components were compounds each having a molecular weight of 358 daltons.

### (3) Sweet Quality Evaluation

The activated carbon treated product (in each of Examples 3 and 4, and Comparative Example 7) prepared in the item (1) was prepared into an aqueous solution (sample) having a concentration of 0.1% by weight/volume. Thereabout, 15 panelists skillful for sensory evaluations made sensory tests.

Specifically, each of the panelists were caused to taste each of the samples. The panelists gave each of the three samples a score of 1, 2 or 3. The scores 1, 2 and 3 were in the order of being from the smallest score toward larger scores about each of off-flavors (total evaluation of bitterness, over-rich flavor, peculiar flavor, astringency, stimulating flavor, non-refreshing flavor (remaining flavor), non-mellowness, and relish) of sweet qualities. The total value of the given scores was then gained. The results are shown in Table 12.

TABLE 12

| | | Caramel | |
|---|---|---|---|
| | | decolorizing | Total of off-flavor |
| | | ability | scores (n = 15) |
| Activated carbon | Example 3 | 94.6% | 32 |
| treated products: | | | |
| | Example 4 | 95.7% | 20 |
| | Comparative | 80.0% | 38 |
| | Example 7 | | |

The results were evaluated through a Kramer test (the number k of the samples=3, the number n of the panelists=15, p<0.05, and a−b=23−37). As a result, Comparative Example 7 was not significantly preferred among the three samples (or had a significant difference from Examples 3 and 4 in that the off-flavors were larger). Example 4 was most significantly preferred among the three species (or had a significant difference from Comparative Example 7 and Example 3 in that the off-flavors were smaller)

### Experimental Example 4: Sweet Quality Evaluation of Agrochemical-Free Siraitia Grosvenorii Extract

Five panelists skillful for sensory evaluations, as subjects, made sensory tests about sweet qualities (8 items) of the activated carbon untreated product (raw material Siraitia Grosvenorii extract) and the activated carbon treated product (Example 4).

### (1) Sample Preparation

### (1-1) Pretreatment

The activated carbon untreated product (raw material Siraitia Grosvenorii extract) and the activated carbon treated product (Example 4) were each supplied to an HPLC under conditions described below, and then the following were each separated and collected: a fraction eluted out before a retention time of 27 minutes (hereinafter referred to as the “(pre-)fraction”); and a fraction eluted out after the retention time of 27 minutes (hereinafter referred to as the “(post-) fraction”). The fractions were each concentrated under a reduced pressure, using a rotary evaporator, and free-dried. At the separating and collecting time, the fractions were each an aqueous solution having a concentration of 5% by weight/weight. At the time of the chromatogram detailed below, the fractions were each a 0.2% aqueous solution.

### [HPLC Conditions]

HPLC apparatus: LC-10ADvp (manufactured by Shimadzu Corp.),
LC column: Nucleosil 100-5C18 (250 mm×20 mm I.D.) (manufactured by GL Sciences Inc.)
LC conditions:
   injected volume: 1 mL,
   column temperature: 40° C.,
   mobile phase: phase A: water, and phase B: acetonitrile,
   0 minutes→10 minutes (isocratic): 80% by volume of phase A, and 20% by volume of phase B,
   10 minutes→50 minutes (linear gradient): “80% by volume of phase A, and 20% by volume of phase B”→“45% by volume of phase A, and 55% by volume of phase B”,
   flow rate: 9.999 mL/minute, and
   detecting wavelength: UV 203 nm.

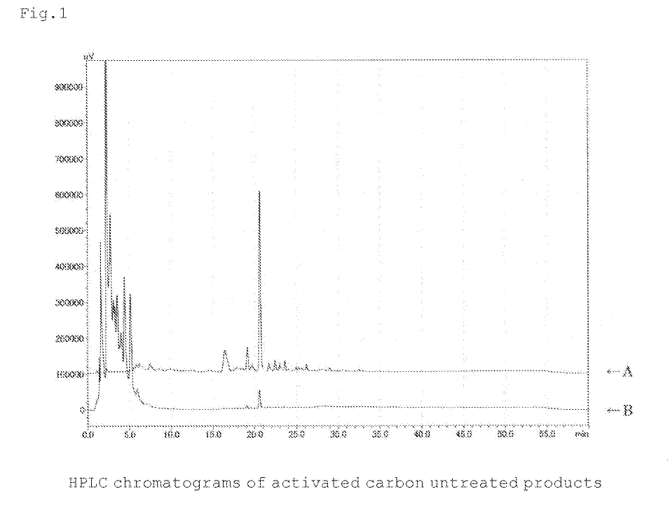

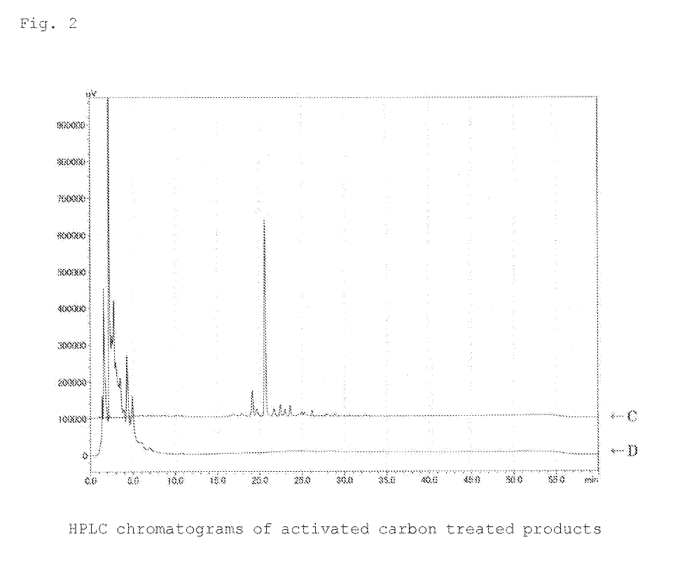

The (pre)-fraction and the (post)-fraction of the activated carbon untreated product, and the (pre)-fraction and the (post)-fraction of the activated carbon treated product were each supplied to an HPLC under conditions described in Experimental Example 3 to give chromatograms (FIG. 1: the activated carbon untreated product, and FIG. 2: the activated carbon treated product). As is understood from a comparison between the chromatograms (in FIGS. 1 and 2) of the individual (post)-fractions, it was verified that by the activated carbon treatment, peak components around a retention time of 16 minutes were removed and decreased.

### (1-2) Sample Preparation

The activated carbon untreated product, the (post)-fraction of the activated carbon untreated product, and the (post)-fraction of the activated carbon treated product each prepared as descried above were each weighed into an amount of 0.05 g. Water was added thereto to adjust the entire amount to 25 g. The resultants were used as sweet quality evaluating samples.

### (2) Sweet Quality Evaluation

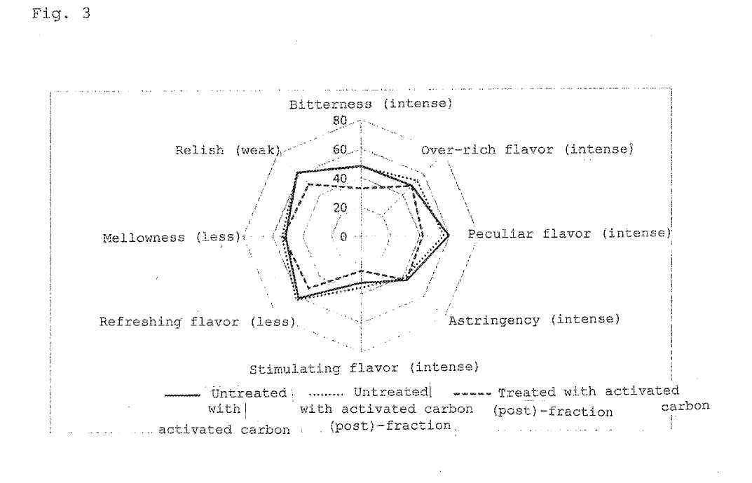

For five healthy examinees as test subjects, the Siraitia Grosvenorii extract, the activated carbon untreated product yielded in Example 4, the (post)-fraction of the activated carbon untreated product, and the (post)-fraction of the activated carbon treated product were each dissolved into water to prepare a 0.2% aqueous solution. About the aqueous solution, a visual analogue scale (VAS) method was used to evaluate sweet qualities (eight items: bitterness, over-rich flavor, peculiar flavor, astringency, stimulating flavor, refreshing flavor, mellowness, and relish) in accordance with absolute scales. The VAS is a method of judging the resultant sensitivity as a point on a straight line, and is widely used for screening examinations of basic tastes such as sweetness (for example, “Investigation on Elderly Persons' Subjective Healthy Feeling Assessment Method”, Psychological Research 2004, No. 3, 89-98: “Construction of Screening Examining Method of Gustatory Sensibility Function for four Basic Tastes”), Stomatognathic Function Journal 20, 115-129, 2014). In the present experiment, a VAS shows a straight line of 100 mm length. The left end thereof represents “weak bitterness”, and the right end thereof represents “strong bitterness”. Each of the test subjects is caused to mark a dot on the line. When the marked dot is positioned at, for example, a site of 50 mm apart from the left end, the test subject is determined to feel a bitterness of a score of 50 (FIG. 3). In other words, as the score value of the bitterness is larger, the bitterness is stronger to be worse for each of the test subjects. Reversely, as the score value of the bitterness is smaller, the bitterness is weaker to be better for sweet qualities. Any one of the testing liquids was handed at random to each of the examinees. About each of the items, the distance from the left end of a VAS line therefor to the marked dot was measured. The average of the respective values of the examinees was plotted on an axis to prepare a radar chart.

The results are shown in FIG. 3.

As illustrated in FIG. 3, between the activated carbon untreated product and the (post)-fraction of the activated carbon untreated product, a difference was hardly recognized in sweet qualities. From this matter, it was verified that the (pre)-fraction of the Siraitia Grosvenorii extract was hardly related to sweet qualities. In the meantime, when the (post)-fraction of the activated carbon treated product was compared with the activated carbon untreated product and the (post)-fraction of the activated carbon untreated product, the former fraction gave a result that the total of the evaluated scores was lowered about the items of bitterness, peculiar sweetness, stimulating flavor, refreshing flavor, and relish. Thus, it was verified that the former fraction had good sweet qualities.

As illustrated in FIGS. 1 and 2, the (post)-fraction of the activated carbon untreated product was different from the (post)-fraction of the activated carbon treated product as to whether the P₁₆ components having a molecular weight of 358 daltons were present or absent. From this matter, the P₁₆ components would be related deeply to a lowering of Siraitia Grosvenorii extracts in sweet qualities (or an increase of off-flavors). According to the matter, the activated carbon treatment in the present invention makes it possible to remove and decrease, effectively from a Siraitia Grosvenorii extract, not only residual agrochemicals but also P₁₆ components related to a lowering in sweet qualities (or an increase of off-flavors). Thus, the treatment is useful for a safe method for preparing a sweetener composition good in sweet qualities.

1. A method for preparing an agrochemical-free Siraitia Grosvenorii extract, comprising the step of treating an agrochemical-containing Siraitia Grosvenorii extract with an activated carbon to remove an agrochemical therein,
wherein the activated carbon used in the activated carbon treatment is at least one activated carbon selected from the group consisting of an activated carbon having an iodine adsorption amount of at least 1500 mg/g, and an activated carbon having a caramel decolorizing ability of at least 85%, and
the recovery ratio of Siraitia Grosvenorii glycosides according to the activated carbon treatment is at least 70%, the glycosides being mogroside V, mogroside IV, 11-oxo-mogroside V, and siamenoside I.

2. The method according to claim 1, wherein the total amount of mogroside V, mogroside IV, 11-oxo-mogroside V, and siamenoside I comprised in the agrochemical-free Siraitia Grosvenorii extract is at least 33% by weight per 100% by weight of the agrochemical-free Siraitia Grosvenorii extract.

3. The method according to claim 1, wherein the agrochemical is at least one selected from the group consisting of dimethomorph, triadimenol, tebuconazole, difenoconazole, metalaxyl, and metalaxyl M.

4. The method according to claim 1, wherein about the agrochemical-free Siraitia Grosvenorii extract, the area of a peak which is detected by an HPLC analysis of the extract under the following conditions and which is around a retention time of 16 minutes is 3% or less of the total area of all peaks detected thereby:
[HPLC Conditions]
column: Kaseisorb LC ODS2000 (150 mm×4.6 mm I.D.)
mobile phase: a mixed liquid of acetonitrile and water,
0 minutes→10 minutes (isocratic): 20% by volume of acetonitrile,
10 minutes→50 minutes (linear gradient): 20% by volume of acetonitrile→55% by volume thereof,
flow rate: 1 mL/minute,
column temperature: 40° C., and
detector: UV 203 nm.

5. An agrochemical-free Siraitia Grosvenorii extract, wherein
the total amount of mogroside V, mogroside IV, 11-oxo-mogroside V, and siamenoside I comprised in the agrochemical-free Siraitia Grosvenorii extract being at least 33% by weight per 100% by weight of the agrochemical-free Siraitia Grosvenorii extract,
the area of a peak which is detected by an HPLC analysis of the extract under the following conditions and which is around a retention time of 16 minutes being 3% or less of the total area of all peaks detected thereby:
[HPLC Conditions]
column: Kaseisorb LC ODS2000 (150 mm×4.6 mm I.D.)
mobile phase: a mixed liquid of acetonitrile and water,
0 minutes→10 minutes (isocratic): 20% by volume of acetonitrile,
10 minutes→50 minutes (linear gradient): 20% by volume of acetonitrile→55% by volume thereof,
flow rate: 1 mL/minute,
column temperature: 40° C., and
detector: UV 203 nm.

6. The agrochemical-free Siraitia Grosvenorii extract according to claim 5, wherein the peak which is detected by the HPLC analysis and which is near the retention time of 16 minutes is a peak of a compound having a molecular weight of 358 daltons.

7. The agrochemical-free Siraitia Grosvenorii extract according to claim 5, wherein the agrochemical is dimethomorph, triadimenol, tebuconazole, difenoconazole, metalaxyl, and metalaxyl M.

8. An agrochemical-free Siraitia Grosvenorii extract, which is prepared by the method of claim 1.

9. A sweetener composition, comprising the agrochemical-free Siraitia Grosvenorii extract of claim 5 and an auxiliary sweetener constituent.

10. The sweetener composition according to claim 9, wherein the auxiliary sweetener constituent is at least one selected from the group consisting of monosaccharides, disaccharides, oligosaccharides, sugar alcohols, non-saccharide sweeteners, and dietary fibers.

11. A food or drink, quasi-drug, cosmetic product or medicine, comprising the sweetener composition recited in claim 9.
